SEQUENCE LISTING (23 sequences)

SEQ ID NO: 1 - protein, 14 aa
ERVRLLERCIYNQE

SEQ ID NO: 2 - protein, 20 aa
GKTRPRFLWQLKFECHFFNG

SEQ ID NO: 3 - protein, 20 aa
LKFECHFFNGTERVRLLERC

SEQ ID NO: 4 - protein, 22 aa
TERVRLLERCIYNQEESVRFDS

SEQ ID NO: 5 - protein, 20 aa
IYNQEESVRFDSDVGEYRAV

SEQ ID NO: 6 - protein, 20 aa
DVGEYRAVTELGRPDAEYWN

SEQ ID NO: 7 - protein, 20 aa
TELGRPDAEYWNSQKDLLEQ

SEQ ID NO: 8 - protein, 25 aa
DLLEQRRAAVDTYCRHNYGVGESFT

SEQ ID NO: 9 - protein, 20 aa
GDTRPRFLWQLKFECHFFNG

SEQ ID NO: 10 - protein, 15 aa
KGNNDESNISFKEKD

SEQ ID NO: 11 - protein, 14 aa
KRAAVDTYCRHNYG

SEQ ID NO: 12 - protein, 14 aa
KRAGVDTYCRHNYG

SEQ ID NO: 13 - protein, 14 aa
KRAVVDTYCRHNYG

SEQ ID NO: 14 - protein, 14 aa
KRASVDTYCRHNYG

SEQ ID NO: 15 - protein, 14 aa
KRAAADTYCRHNYG

SEQ ID NO: 16 - protein, 14 aa
KRAAGDTYCRHNYG

SEQ ID NO: 17 - protein, 14 aa
KRAVLDTYCRHNYG

SEQ ID NO: 18 - protein, 14 aa
KRAAVDSYCRHNYG

SEQ ID NO: 19 - protein, 14 aa
KRAAVDDYCRHNYG

SEQ ID NO: 20 - protein, 14 aa
KRAAVDVYCRHNYG

SEQ ID NO: 21 - protein, 14 aa
KRAAVDTYSRHNYG

SEQ ID NO: 22 - protein, 14 aa
KRAAVDTYARHNYG

SEQ ID NO: 23 - protein, 14 aa
KRAAVDTYNRHNYG

(19) United States
(12) Patent Application Publication
(10) Pub. No.: US 2002/0155117 A1
(43) Pub. Date: Oct. 24, 2002

(54) METHOD FOR DETECTING ORGAN ALLOGRAFT REJECTION AND USES THEREOF

(76) Inventor: NICOLE SUCIU-FOCA, CLIFFSIDE PARK, NJ (US)

Correspondence Address: JOHN P WHITE, COOPER & DUNHAM, 1185 AVENUE OF THE AMERICAS, NEW YORK, NY, 10036

(21) Appl. No.: 08/677,599

(22) Filed: Jul. 8, 1996

Related U.S. Application Data
(60) Provisional application No. 60/014,820, filed on Apr. 4, 1996.

(51) Int. Cl. C12Q 1/00; A61K 39/00

(52) U.S. Cl. 424/185.1; 424/198.1

(57) ABSTRACT

This invention provides for a method for monitoring allograft rejection in a transplant recipient which includes: (a) obtaining peripheral blood lymphocytes from the recipient; (b) incubating the lymphocytes with at least one synthetic allopeptide which corresponds to an immunogenic hypervariable epitope of a mismatched donor HLA-DR antigen under suitable conditions, and (c) contacting the incubate with a marker for T-cell activation to determine whether or not the incubate contains activated T-cells, thereby monitoring allograft rejection in the recipient.

This application claims the benefit of U.S. Provisional Application No. 60/014,820, filed Apr. 4, 1996, the contents of which are hereby incorporated by reference into the present application.

The invention disclosed herein was made with Government support under NIH Grant No. RO1-A125210-08 from the Department of Health and Human Services and Grant No. IM-694 from the American Cancer Society. Accordingly, the U.S. Government has certain rights in this invention.

### BACKGROUND OF THE INVENTION

Throughout this application, various publications are referenced by author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

The rejection of allografts results from two distinct forms of antigen presentation: (Sherman and Chattopadhyan, 1993) donor antigen presenting cells (APC) can activate directly the response of host T cell; and (Shoskes and Wood, 1994) host APC can present peptides derived from the processing of allogeneic MHC molecules to autologous T cells, engaging them into the indirect pathway of allorecognition. (Sherman and Chattopadhyan, 1993; Shoskes and Wood, 1994).

All T cell recognition, including self-tolerance and allorecognition, involves both the MHC molecule and its associated peptide ligand. An important step in understanding alloreactivity is understanding T cells can engage and respond to allogenic MHC molecules directly. Direct presentation has been shown to be responsible for the proliferative response to alloantigens seen in vitro and events leading to early acute rejection of MHC-mismatched skin grafts. Indirect presentation is another pathway for the presentation of MHC alloantigens. In this pathway, the allogeneic MHC molecules are processed and presented by recipient antigen-presenting cells (APCs). (For reviews see, Sherman and Chattopadhyay, 1993 and Shoskes and Wood, 1994).

The success rate of organ transplantation has increased considerably during the past decade with substantial improvements in one year graft and patient survival. At the same time, however, it has become clear that a significant proportion of grafts fail, primarily because of progressive and irreversible host-immunological attack, despite continued administration of immunosuppression. Large statistics demonstrate, that although short-term results have improved, the rate of decline of long functioning grafts has not changed over time regardless of changes in immunosuppression. Roughly only 60% of kidney, heart or liver allografts are still functioning after 5 years (Harris et al. 1990). Late graft failures are attributed to the uncontrollable progression of chronic rejection, a phenomenon involving both the cellular and humoral arm of the immune response.

Chronic rejection is often described as “transplant atherosclerosis” and is an immunologic phenomenon accompanied by the presence of T cells and macrophages in graft atherosclerotic lesions, and of immunoglobulin and complement deposition in affected vessel walls. The production of anti-donor HLA antibodies precedes and accompanies the chronic rejection process and intra-arterial infusion of donor-specific anti-sera induces the characteristic histological lesions. Anti-HLA antibodies bind to graft endothelial cells mediating complement dependent cytotoxicity and ADCC. Plasmapheresis has been used with mixed success for lowering the titer of circulating antibodies and slowing down the progression of humoral rejection. One of the consequences of transplant failure is the increasing number of “highly sensitized patients” who have rejected a previous transplant, and consequently have developed alloantibodies. This renders the task of finding a suitable donor for retransplantation substantially more difficult. The risk of opportunistic infections and malignancies is also increased in such patients, because of the large doses of immunosuppressive therapy which they receive (Wasfie, et al. 1990).

### SUMMARY OF THE INVENTION

This invention provides for a method for monitoring allograft rejection in a transplant recipient which includes: (a) obtaining peripheral blood lymphocytes from the recipient; (b) incubating the lymphocytes with at least one synthetic allopeptide which corresponds to an immunogenic hypervariable epitope of a mismatched donor HLA-DR antigen under suitable conditions, and (c) contacting the incubate with a marker for T-cell activation to determine whether or not the incubate contains activated T-cells, thereby monitoring allograft rejection in the recipient.

### BRIEF DESCRIPTION OF THE FIGURES

FIGS. 1A and 1B. Reactivity of TCLs to the immunizing peptides and to r DR1 proteins. The TCL LZ-anti-pp 1-20 (FIG. 1B) and TCL LZ-anti-pp 21-42 (FIG. 1B) were tested for reactivity to various concentrations of rDR1 protein and pp 1-20 pr pp 21-42 using 2×10⁴ responding cells and 5×10⁴ irradiated autologous APCs per well. Cultures were labeled after 48 hours and harvested 18 hours later. Results are expressed as mean cpm of triplicate cultures. The standard deviation of measure was less than 10%.

FIGS. 2A and 2B. HLA-DR restriction of peptide recognition by TCLs. The TCL LZ-anti-pp 1-20 (FIG. 2A) and TCL LZ-anti-pp 21-42 (FIG. 2B) were stimulated with 1 mM peptide (pp 1-20 for LZ-anti-pp 1-20 and pp 21-42 for LZ-anti-pp 21-42) in the presence of autologous and allogeneic APCs sharing with the responder one or no DR antigen. Assays were done as described in the description of FIGS. 1A and 1B.

FIG. 3. Determination of the core epitope of the dominant determinant of DR1 molecule. TCL LZ-anti-pp 21-42 was stimulated in individual cultures with nine overlapping 14-mer peptides (1 mM) moving along residues 21-42 in single residue steps. The reactions were set up as described in the description of FIGS. 1A and 1B.

FIG. 4. Suppression of TCL reactivity by high concentrations of antigen. The proliferative response of TCL LZ-anti-pp 21-42 was tested as described in the description of FIGS. 1A and 1B using different concentrations of pp 22-35. The response was abolished at a concentration 27 mM.

FIG. 5. CML activity of graft infiltrating lymphocytes. Il-2 expanded lymphocytes from the day 55 (filled column) and 82 (open column) biopsies were tested for cytotoxic activity against PHA activated lymphocytes from the donor, recipient and HLA-disparate control. The effector to target cell ratio was 10:1. All reactions were done in triplicate. Results given are the mean cpm.

FIG. 6. Suppression of in vivo activated T cells by high doses of antigen. PBMCs obtained from patient PI on day 55 were expanded in IL-2 for 14 days and then tested in LDA (limiting dilution analysis) for reactivity to pp 22-35 at concentrations of 0, 1 and 15 mM in the presence of autologous APCs.

FIG. 7. Association between acute rejection and Th reactivity to donor allopeptides. Serial blood samples were obtained from heart transplant recipients and tested in LDA for reactivity to donor HLA-DR peptides. LDA was considered positive if more than 1.5×10⁻⁶ Th reactive to one or both of the donor's HLA-DR antigens were detected. Rejection was monitored by EMB and considered to be present when graded histologically 1B or higher. Incidence of rejection one month following LDA was calculated by the product-limit method.

FIGS. 8A, 8B, 8C and 8D. Intermolecular alloantigen spreading during the course of multiple acute rejection episodes. PBMCs were collected from each patient at various times following transplantation and tested in LDA for reactivity against peptides corresponding to each of the mismatched HLA-DR antigens of the donor. Th frequency was determined as described in Zhang et al., 1994. Grading of each biopsy at the time when the blood was collected is indicated.

### DETAILED DESCRIPTION OF THE INVENTION

This invention provides for a method for monitoring allograft rejection in a transplant recipient which includes: (a) obtaining peripheral blood lymphocytes from the recipient; (b) incubating the lymphocytes with at least one synthetic allopeptide which corresponds to an immunogenic hypervariable epitope of a mismatched donor HLA-DR antigen under suitable conditions, and (c) contacting the incubate with a marker for T-cell activation to determine whether or not the incubate contains activated T-cells, thereby monitoring allograft rejection in the recipient.

The synthetic allopeptide may include at least a portion of a peptide chosen from the group: DRβ1*0101, DRβ1*0102, DRβ1*0301, DRβ1*0302, DRβ1*0401, DRβ1*0102, DRβ1*0403, DRβ1*0404, DRβ1*0405, DRβ1*0407, DRβ1*0408, DRβ1*0701, DRβ1*0801, DRβ1*0802, DRβ1*0803, DRβ1*0804, DRβ1*0901, DRβ1*1001, DRβ1*1101, DRβ1*1102, DRβ1*1104, DRβ1*1201, DRβ1*1301, DRβ1*1302, DRβ1*1303; DRβ1*1401, DRβ1*1402, DRβ1*1502, DRβ1*1503, DRβ1*1601, or DRβ1*1602.

The synthetic allopeptide may include a portion of a hypervariable region of an HLA-DR molecule. The portion of the HLA-DR hypervariable region may include amino acids 1-19, amino acids 20-40 or amino acids 41-80.

The allograft may include a heart, a kidney, a liver, skin, bone, bone marrow, an eye, hair, or a lung.

The determination of whether or not the incubate contains activated T-cells may include detecting T-cell blastogenesis, detecting lymphokine production by the T-cell or detecting expression of an activation marker by the T-cell.

The activation marker may be a monoclonal antibody. The monoclonal antibody may be CD69, CD64, LDA1, NDA4 or γIF. The activation marker may also be any early activation marker of T cells which measures specific early activation to the alloantigen (donor antigen). The early activation marker may measure tyrosine phosphorylation.

The synthetic allopeptide may include: amino acids 21-39 of a DRβ1*0101 molecule wherein the recipient expresses a DRβ1*1104 haplotype or a DRβ1*1101 haplotype; amino acids 1-19 of a DRβ1*0301 molecule wherein the recipient expresses a DRβ1*1101 haplotype; amino acids 62-80 of a DRβ1*0401 molecule wherein the recipient expresses a DRβ1*0701 haplotype; amino acids 21-42 of DRβ1*1101 molecule wherein the recipient expresses a DR3 haplotype; amino acids 21-40 of DRβ1*0401 molecule wherein the recipient expresses a DRβ1*0701; amino acids 1-19 of a DRβ1*1301 molecule wherein the recipient expresses a DRβ1*0701 haplotype; amino acids 1-19 of a DRβ1*1501 molecule wherein the recipient expresses a DRβ1*0401 haplotype and a DRβ1*1104 haplotype, or amino acids 21-29 of a DRβ1*1503 molecule wherein the recipient expresses a DRβ1*1501 haplotype.

One embodiment of the present invention provides for a method for treating allograft rejection in a transplant recipient which includes administering to the recipient a mixture of soluble MHC derived from the recipient and at least one synthetic allopeptide which corresponds to an immunogenic hypervariable epitope of a mismatched donor HLA-DR antigen thereby treating allograft rejection in the recipient.

Another embodiment of the present invention is a method for treating allograft rejection in a recipient which includes:

(a) determining whether or not allopeptide reactive T-cells are present in the recipient, and if such cells are present, and

(b) administering to the recipient a mixture of soluble MHC derived from the recipient and an allopeptide which comprises ERVRLLERCIYNQE, so as to treat allograft rejection in the subject.

The present invention also provides a method for inhibiting chronic allograft rejection in a recipient which includes administering to the recipient a peptide antigen corresponding to an epitope on a donor allo-MHC molecule so as to induce tolerance in the recipient and thereby inhibit chronic allograft rejection in the recipient.

The peptide antigen may be administered to the recipient at a dose of about 10 micrograms per million lymphocyte cells of recipient. The dose may also be from about 5 micrograms/million lymphocytes to about 200 micrograms/million lymphocytes.

Another embodiment of the present invention is a vaccine against an allopeptide which includes a synthetic T-cell receptor peptide which is designed to react against a mismatched MHC-DR allele of an organ donor. The mismatched MHC-DR allele may include: DRβ1*0101 wherein the recipient expresses a DRβ1*1104 haplotype or a DRβ1*1101 haplotype; DRβ1*0301 wherein the recipient expresses a DRβ1*1101 haplotype; DRβ1*0401 wherein the recipient expresses a DRβ91*0701 haplotype; DRβ1*1301 wherein the recipient expresses a DRβ1*0701 haplotype; DRβ1*1501 wherein the recipient expresses a DRβ1*0401 haplotype and a DRβ1*1104 haplotype, or DRβ1*1503 wherein the recipient expresses a DRβ1*1501 haplotype.

Another embodiment of the present invention is a method for detecting chronic allograft rejection in a subject which includes identification of an immune response to an alloantigen in the subject which comprises:(a) obtaining a blood sample from the subject which sample contains peripheral blood T-cells;(b) incubating the PBMCs in a mixture including an allopeptide, which allopeptide comprises GKTRPRFLWQLKFECHFFNG, LKFECHFFNGTERVRLLERC, TERVRLLERCIYNQEESVRFDS, IYNQEESVRFDSDVGEYRAV, DVGEYRAVTELGRPDAEYWN, TELGRPDAEYWNSQKDLLEQ or DLLEQRRAAVDTYCRHNYGVGESFT under conditions suitable for peripheral blood T-cell activation; and (c) evaluating the capacity of the peripheral blood T-cell to react against the allopeptide indicating an immune response of the subject against the alloantigen, thus detecting allograft rejection in the subject. The evaluating the capacity in step (c) may include detecting T-cell blastogenesis, detecting lymphokine production by the T-cell or detecting expression of an activation marker by the T-cell. The activation marker may be a monoclonal antibody. The activation marker may be CD69 or CD64. The CD69 or CD64 may be manufactured by Becton-Dickenson.

In one embodiment of this invention, the allopeptide may include at least a portion of a sequence of a dominant determinant of an allogenic MHC molecule of an allograft donor. The sample may include about 5 cc to about 20 cc of blood. The allograft may include a heart, a kidney, a liver, skin or a lung.

Another embodiment of the present invention is a method for detecting chronic allograft rejection in a subject which includes identification of an immune response to an alloantigen in the subject which includes:(a) obtaining a blood sample from the subject which sample contains peripheral blood T-cells; (b) incubating the PBMCs in a mixture including an allopeptide, which allopeptide may include ERVRLLERCIYNQE under conditions suitable for peripheral blood T-cell activation; and (c) evaluating the capacity of the peripheral blood T-cell to react against the allopeptide indicating an immune response of the subject against the alloantigen, thus detecting allograft rejection in the subject. The evaluation of the capacity in step (c) may include detecting T-cell blastogenesis, detecting lymphokine production by the T-cell or detecting expression of an activation marker by the T-cell.

The present invention also provides for a method for screening for allograft rejection in a subject which includes: (a) obtaining a blood sample from the subject which sample contains peripheral blood T-cells; (b) incubating the peripheral blood T-cells in a reaction mixture including donor-matched synthetic allopeptides which allopeptides comprise GKTRPRFLWQLKFECHFFNG, LKFECHFFNGTERVRLLERC, TERVRLLERCIYNQEESVRFDS, IYNQEESVRFDSDVGEYRAV, DVGEYRAVTELGRPDAEYWN, TELGRPDAEYWNSQKDLLEQ or DLLEQRRAAVDTYCRHNYGVGESFT under conditions suitable for peripheral blood T-cell activation; and (c) determining whether or not the peripheral blood T-cells are activated thereby detecting allograft rejection in the subject.

Another embodiment of the present invention is a method for screening for allograft rejection in a subject which includes: (a) obtaining a blood sample from the subject which sample contains peripheral blood T-cells;(b) incubating the peripheral blood T-cells in a reaction mixture including a donor-matched synthetic a lopeptide which allopeptide comprises ERVRLLERCIYNQE under conditions suitable for peripheral blood T-cell activation; and (c)determining whether or not the peripheral blood T-cells are activated thereby detecting allograft rejection in the subject.

The present invention further provides for a method for treating allograft rejection in a subject which includes: (a) determining whether or not allopeptide reactive T-cells are present in the periphery of the subject, and if such cells are present; and (b) administering to the subject a mixture of soluble MHC derived from the subject and an allopeptide chosen from the group CKTRPRFLWQLKFECHFFNG, LKFECHFFNGTERVRLLERC, TERVRLLERCIYNQEESVRFDS, IYNQEESVRFDSDVGEYRAV, DVGEYRAVTELGRPDAEYWN, TELGRPDAEYWNSQKDLLEQ or DLLEQRRAAVDTYCRHNYGVGESFT, so as to treat allograft rejection in the subject.

The present invention further provides for a method for treating allograft rejection in a subject which includes: (a) determining whether or not allopeptide reactive T-cells are present in the periphery of the subject, and if such cells are present; and (b) administering to the subject a mixture of soluble MHC derived from the subject and a allopeptide which includes ERVRLLERCIYNQE, so as to treat allograft rejection in the subject.

A further embodiment of the present invention is a method for inhibiting chronic rejection of an allograft in a subject including controlled activation of alloreactive T helper cells which includes: (a) determining the dominant epitope on an allo-MHC molecule of an allograft donor and the subject; and (b) administering to the subject a mixture including soluble MHC derived from the subject and a synthetic peptide chosen from the group GKTRPRFLWQLKFECHFFNG, LKFECHFFNGTERVRLLERC, TERVRLLERCIYNQEESVRFDS, IYNQEESVRFDSDVGEYRAV, DVGEYRAVTELGRPDAEYWN, TELGRPDAEYWNSQKDLLEQ or DLLEQRRAAVDTYCRHNYGVGESFT so as to inhibit chronic rejection of the allograft in the subject.

The present invention further provides for a method for inhibiting chronic rejection of an allograft in a subject including controlled activation of alloreactive T helper cells which includes:(a) determining the dominant epitope on an allo-MHC molecule of an allograft donor and the subject; and (b) administering to the subject a mixture including soluble MHC derived from the subject and a synthetic peptide, which peptide includes ERVRLLERCIYNQE so as to inhibit chronic rejection of the allograft in the subject.

The present invention further provides for a vaccine against allopeptides which includes synthetic T cell receptor peptides which are designed to react against a mismatched MHC-DR allele of an organ donor.

Another embodiment of the present invention is a vaccine against chronic allograft rejection in a subject which includes synthetic T cell receptor peptide which peptide includes ERVRLLERCIYNQE.

Another embodiment of the present invention is a kit for detecting chronic allograft rejection in a subject which includes: (a) at least one allopeptide chosen from the group GKTRPRFLWQLKFECHFFNG, LKFECHFFNGTERVRLLERC, TERVRLLERCIYNQEESVRFDS, IYNQEESVRFDSDVGEYRAV, DVGEYRAVTELGRPDAEYWN, TELGRPDAEYWNSQKDLLEQ or DLLEQRRAAVDTYCRHNYGVGESFT; and (b) a means of determining activation of T-cells of the subject.

A further embodiment of the present invention is a kit for detecting chronic allograft rejection in a subject which includes: (a) at least one allopeptide which allopeptide includes ERVRLLERCIYNQE; and(b) a means of determining activation of T-cells of the subject.

As used herein, “alloantigen” encompasses any antigen recognized by different individuals of the same species.

As used herein, “allotype” encompasses a genetically different antigenic determinant on a protein of an individual of the same species.

As used herein, “allogeneic” encompasses a genetically different phenotype present in nonidentical individuals of the same species. Allogeneic examples include blood group phenotypes and immunoantigeneic allotypes.

As used herein, an “allograft” encompasses a transplant of an organ, tissue, bodily fluid or cell from one individual to a genetically nonidentical individual of the same species.

This invention is illustrated in the Experimental Detail section which follows. These sections are set forth to aid in an understanding of the invention but are not intended to, and should not be construed to, limit in any way the invention as set forth in the claims which follow thereafter.

### EXPERIMENTAL DETAILS

### EXAMPLE 1

### Indirect Recognition of Donor MHC-Class II Antigens in Human Transplantation

Abbreviations

APC—antigen presenting cell; DAF—decay accelerating factor; MHC—major histocompatibility complex; HLA—human lymphocyte antigen; LDA—limiting dilution analysis; PBMC—peripheral blood mononuclear cell; Tc—cytotoxic T cell; TCL—T cell line; Th—T helper cell; TCR—T cell receptor.

Introduction

To investigate the role of the indirect pathway of recognition in human allograft rejection, the dominant T cell determinant of the HLA-DRβ1*0101 molecule presented by the DRβ1*0101 antigen has been mapped. A synthetic peptide (pp 22-35) corresponding to the sequence of the dominant peptide determinant was used for testing the frequency of in vivo activated T cells in the graft and in the periphery. Drβ1*0101 positive patients carrying a heart allograft mismatched for the HLA-DR1 antigen showed no reactivity to pp 22-35 during quiescence. However, IL-2 responsive T cells, which were pp 22-35 specific, were found in the circulation prior to and at the time of acute and chronic rejection. The response of in vivo and in vitro activated T cells was inhibited at high concentrations of peptide 22-35. This data suggests that indirect recognition plays an important role in allograft rejection and that it can be abolished by high zone tolerance induction.

The indirect pathway explains the extraordinary strength of alloreactivity since the many different peptides bound to the cleft of allogeneic MHC class I and class II molecules may trigger the activation of alloreactive (CD8⁺ and CD⁺) T cells, respectively. This pathway, however, does not explain the development of anti-HLA antibodies which mediate humoral rejection of the acute vascular and chronic type. In general, such anti-donor HLA antibodies are IgG in nature suggesting theat T helper (Th) cells, which recognize processed forms of the alloantigen, provide the lymphokines required for the growth and maturation of B cells that produce the corresponding alloantibodies (Suciu-Foca et al., 1991a; Suciu-Foca et al., 1991b). Indirect allorecognition may, therefore, activate the humoral arm of the immune response playing an important role in allograft rejection. In addition, T cells engaged in the indirect pathway may contribute to Delayed Type Hypersensitivity (DTH) and cytotoxic T cell (Tc) responses (Shoskes and Wood, 1994; Lee et al., 1994). Evidence to this effect was provided by experiments showing that immunization with allopeptides accelerates rejection of subsequent grafts which express the intact antigen, and that T lymphocytes from alloimmunized animals react specifically to synthetic peptides derived from the structure of donor MHC-class II molecules (Lee et al., 1994; Benichou et al., 1992; Fangmann et al., 1992; Dalchau et al., 1992; Parker et al., 1992; Sayegh et al., 1992; and Gallon et al., 1995).

Studies in the human system have shown that indirect recognition of peptides derived from allogeneic MHC molecules is MHC restricted and that it involves a limited T cell receptor (TCR) repertoire (de Koster et al., 1989; Chen et al., 1990; Liu et al., 1992; Liu et al., 1993a; and Liu et al., 1993b). Furthermore, studies of the relative contribution of direct and indirect recognition to alloreactivity showed that Th cells participating in the latter have a precursor frequency which is about 100 fold lower than that of the Th engaged in direct recognition (Liu et al., 1992).

These experiments, however, were performed by immunizing T cells from healthy individuals with soluble HLA alloantigens or with synthetic allopeptides. No evidence has been provided as yet that T cells from transplant recipients are sensitized to donor allopeptides and that they may participate in graft rejection. This possibility has been explored by testing the capacity of T cells from patients' peripheral blood and graft biopsies to react against the dominant epitope of a mismatched HLA-DR antigen. T cells from individuals carrying the DRβ1*0101 allele recognize peptide 21-42 as the dominant immunogenic peptide of the DRβ1 molecule presented by the DR11 antigen (Liu et al., 1992). Mapping the immunogenic core of pp-21-42 and testing T cell reactivity to this peptide has been done in patients who carried the DR 11 antigen and received a heart transplant mismatched for DR1. These experiments show for the first time that indirect recognition of donor MHC-class II takes place in the periphery and in the graft and contributes to both acute and chronic rejection.

Materials and Methods

Patients

All donors and recipients were typed for HLA-Class I antigens by conventional serology and for HLA-Class II antigens by genomic typing of in vitro amplified DNA with sequence specific oligonucleotide probes. These studies included three heart allograft recipients who carried the Drβ1*0101 allele and were transplanted with a graft which was HLA-DR mismatched only for Dr1*0101. All patients were monitored for rejection by endomyocardial biopsies (Winters, 1991). Biopsies were examined histologically and for lymphocyte growth in the presence of recombinant IL-2 (rIL-2) (Boehringer Mannheim, Indianapolis Ind.) at 5 u/ml. PBMCs were obtained from the patient at the time of each biopsy.

Peptide Synthesis

Peptides were synthesized with an automated peptide synthesizer (430A, Applied Biosystems, Inc., Foster City, Calif.), using modified Merrifield chemistry, as previously described (Liu et al., 1992). Seven partially overlapping peptides, corresponding to residues 1-20, 11-30, 21-42, 31-50, 43-62, 51-70 and 66-90 of Drβ1*0101 chain, and nine overlapping 14-mers peptides moving along residues 21-42 in single steps were synthesized (Bell et al., 1989).

Production and Purification of Soluble DR1 Protein

Recombinant DR1 (rDR1) was constructed by the method of Scheirle et al. (Scheirle et al., 1992). The cDNA encoding the β-chain of Drβ1*0101 and decay accelerating factor (DAF) were prepared from the lymphoblastoid cell line 10w9004. The external domains of the DRβ gene and the DAF gene were PCR-amplified using primers and amplification conditions previously described (Scheirle et al., 1992). The Drβ1-DAF fusion product was cloned with the TA Cloning System (Invitrogen, San Diego, Calif.) and the sequence confirmed. The construct was subcloned into the pVL1392 baculovirus expression vector (Invitrogen, San Diego, Calif.) and was transferred to the genome of the AcNP virus by homologous recombination. The recombinant virus (BV-DRβ1*0101-DAF) was plaque purified and insertion confirmed by PCR. The recombinant baculovirus BV-DRA-DAF contains the sequences encoding the α-chain of DR. The SF9 insect cells (Invitrogen, San Diego, Calif.) were coinfected at 10⁶/ml with BV-DRA-DAF and BV-DRβ1*0101-DAF. Surface expression of DR1 molecules on infected SF9 cells was monitored by flow cytometry using the anti-HLA DR mAb L243 (ATCC, Rockville, Md.). Ninety-six hours following infection, cells were washed two times with PBS and lysed with detergent in the presence of protease inhibitors. After preclearing, the rDR1 protein was immunoprecipitated with mAb L243 coupled to Sepharose 4B at 4° C. for 3 hours. The column was then incubated with phosphoinositol phospholipase c (2 u) for 2 hours at 27° C. The elution of bound rDR1 was performed using 50 mM diethylamine and 150 mM NaCl, pH 11.5. The purity of the protein was greater than 95% as determined by SDS-PAGE and silver staining. The purified rDR1 protein was dialyzed against RPMl 1640 before use as antigen (Harris et al., 1992).

Development of Allo-peptide Specific T Cell Lines for Epitope Mapping. PBMCs (at 10⁶/ml) from a healthy male (LZ) with HLA-DRβ1*1101/1201 genotype were primed in individual cultures (Liu et al., 1992) with each or the following seven DR1 peptides(5 μg/ml) spanning the first domain of the Drβ1*0101 chain:

| | |
|---|---|
| pp 1-20 | G D T R P R F L W Q L K F E C H F F N G (Seq. I.D. No. 9) |
| pp-11-30 | L K F E C H F F N G T E R V R L L E R C (Seq. I.D. No. 3) |
| pp 21-42 | T E R V R L L E R C I Y N Q E E S V R E D S (Seq. I.D. No. 4) |
| pp 31-50 | I Y N Q E E S V R F D S D V G E Y R A V (Seq. I.D. No. 5) |
| pp 43-62 | D V G E Y R A V T E L G R P D A E Y W N (Seq. I.D. No. 6) |
| pp 51-70 | T E L G R P D A E Y W N S Q K D L L E Q (Seq. I.D. No. 7) |
| pp 66-90 | D L L E Q R R A A V D T Y C R H N Y G V G E (Seq. I.D. No. 8) |
| | S F T |

T cells were grown in 24 well plates (Nunc, Inc., Naperville, Ill.) in RPM1 1640 medium supplemented with 10% human AB serum, 2 μM L-glutamine, and 50 μg/ml gentamicin (Gibco, Grand Island, N.Y.). On day 10, rIL-2 was added at 5 μ/ml. Fourteen days after priming, T cells were collected, washed and restimulated at 10⁶/ml with antigen, in culture medium containing rIL-2 (20 u/ml) and irradiated (3000 rad) autologous PBMCs (2×10⁶/ml). Following three stimulations, two TCLs, named LZ-anti-pp1-20 and LZ-anti-pp21-42, were obtained.

Proliferation Assay

Responding T cells (2×10⁴/well) were cultured with irradiated APCs of responder or stimulator origin (5×10⁴/well) in round bottom microtiter plates (Nunc Inc., Naperville, Ill.). Antigens were added to the cultures at the concentrations indicated. [³H] thymidine (1 μCi/well) was added after 48 hours, and the cultures were harvested 18 hours later. [³H] thymidine incorporation was measured in an LK Betaplate liquid scintillation counter (Wallace, Inc., Gaithersburg, Md.). Mean cpm in triplicate cultures and standard deviation of the mean were calculated.

CML Assay

Spleen cells from the donor and PBMCs from the recipient and from HLA disparate controls were stimulated with 2 μg/ml PHA (Sigma Chemical Co., St. Louis, Mo.) 2-3 days before use as targets for the CML assay. Lymphoblasts (2×10⁶/ml) were labeled with Cr (0.1 mCi/ml) at 37° C. incubator. After 5 hours, the plates were spun again and 0.1 ml supernatant from each well was transferred to a glass tube and counted in a γ counter (Packard, Downers Grove, Ill.). Specific lysis was determined using the formula: [100×(Experimental release-spontaneous release)/(maximum release-spontaneous release)].

Limiting Dilution Analysis (LDA) of Th Frequency in the Periphery

The LDA study of Th frequency in the periphery was performed using the method described by Zhang et al. (Zhang et al., 1994) with some modifications. PBMCs were plated at 2×10⁴, 1×10⁴ and 5×10³ cells per well (24 wells for each concentration) in 0.1 ml medium containing 40 U/ml of rIL-2. On day 7, 0.1 ml fresh medium with rIL-2 was added to each well. After 14 days, each culture was split into 3 aliquots: one was cultured with autologous (patient) irradiated PBMC (5×10⁴/well), the second with autologous PBMC plus pp 22-35, and the third with donor spleen cells (5×10⁴/well). The cultures were pulsed with [³H] thymidine (1 Ci/well) after 48 hours and harvested 18 hours later.

Limiting Dilution Analysis of Th Frequency Among Lymphocytes Infiltrating the Graft

Biopsy tissue was washed twice with medium supplemented with 5 u/ml rIL-2. Cell growth was checked everyday. The medium was changed twice a week. T cells infiltrating the graft were further expanded in medium with rIL-2 for 7-14 days. When the cell number reached 1×10⁶, they were harvested and plated at 400, 200 and 100 cells per well in the presence of autologous PBMC (5×10⁴/per well) in 24 replicate cultures. Cultures were maintained and assayed for reactivity as described above. The precursor frequency of Th cells recognizing pp 22-35 and donor spleen cells was analyzed by Poisson statistics (Liu et al., 1992; and Zhang et al., 1994).

Results

Mapping of Dominant and Cryptic Imminogenic Peptides of the DR1 Molecule Presented by the DR11 Antigen

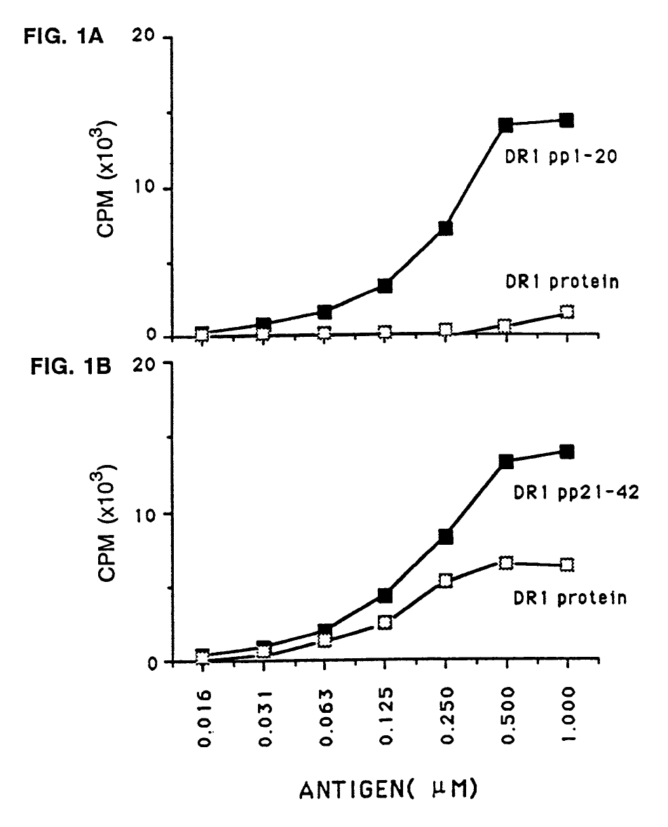

To monitor indirect recognition in allograft recipients, a determination was made as to whether T cell recognize one or more peptides resulting from the processing of an allogeneic MHC molecule by responder APCs. For this, in vitro PBMC were immunized from a responder (LZ), expressing HLA-DR β1*1101/1201, with seven partially overlapping peptides corresponding to the first domain of the Drβ1*0101 molecule. Only two of the seven peptides used, one corresponding to residue 1-20 (pp 1-20) and the other to residue 21-42 (pp 21-42) elicited blastogenesis in the presence of autologous APCs. The resulting T cell lines were tested for their ability to recognize native rDR1 molecule at various protein concentrations. The TCL immunized to pp 1-20 was stimulated strongly by this peptide yet reacted poorly to rDR1 protein presented by autologous APCs (FIGS. 1A and 1B). This indicates that pp 1-20 represents a cryptic immunogenic peptide derived from the DR1 molecule which is not efficiently processed by the responder's APCs.

In contrast, the TCL immunized against pp 21-42 reacted vigorously both to the immunizing peptide and to rDR1 protein (FIG. 1B). Residue 21-42, therefore, comprises the dominant determinant of the DR1 molecule recognized by this responder.

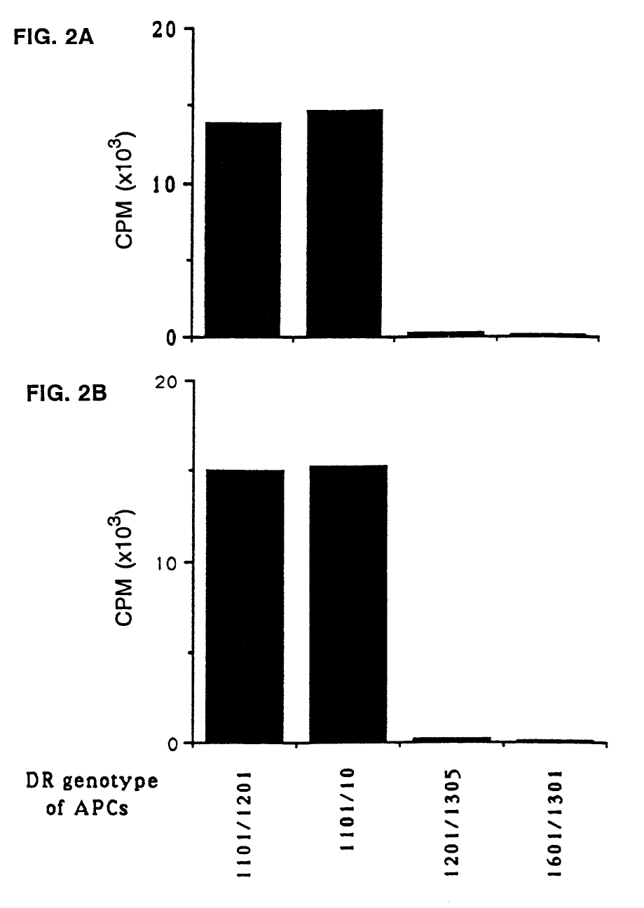

Restriction experiments showed that both TCLs (anti-pp 1-20 and anti-pp 21-42) recognized the immunizing peptide only when presented by APCs carrying the Drβ1*1101 allele. APCs sharing with the responder the Drβ*1201 allele or APCs expressing different HLA-DR antigens were unable to present pp 1-20 or pp 21-42 to the corresponding TCLs (FIGS. 2A and 2B).

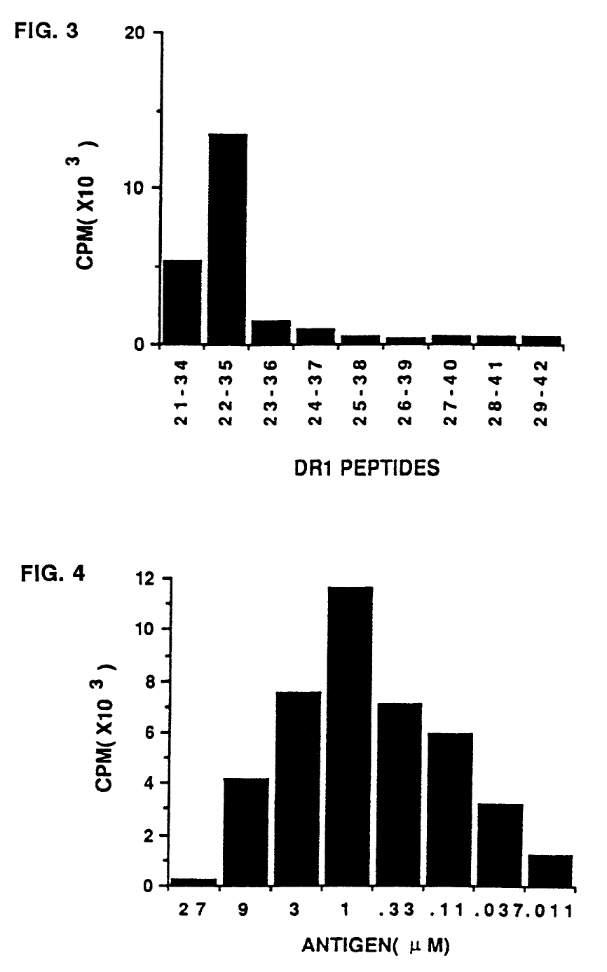

To identify the dominant determinant core of the DR1 molecule nine overlapping 14-mer peptides moving along residues 21-42 were synthesized in single residue steps. Peptide 22-35 stimulated strongly the response of TCL anti-pp 21-42 in the presence of autologous APCs. No other peptides elicited blastogenesis except for pp 21-34 which was, however, less stimulatory (FIG. 3). Hence, residues 22-35 comprise the core of the dominant DR1 determinant recognized by Drβ1*1101 responders.

Suppression of the Reactivity of TCL LZ-anti-pp 21-42 with High Doses of the Peptide Antigen

The immune response of mature T cells can paradoxically be suppressed by high doses of antigen (Suzuki et al., 1988; and Critchfield et al, 1994). The possibility of suppressing indirect allorecognition was explored by testing the reactivity of TCL LZ-anti-pp 21-42 to increasing concentrations of pp 22-35 in the presence of autologous APC. The reactivity of the line started to decline when the concentration of the peptide was increased above 3 μM (FIG. 4). Complete suppression was achieved at the concentration of 27 μM. This result supports the notion that indirect recognition is amendable to suppression by use of high concentrations of dominant allopeptide.

In order to show that the suppression of the T cell response at high concentrations of pp 22-35 was specifically caused by this peptide, and not by an artifact of the culture conditions, the reactivity of TCL-ZL against pp 22-35 was tested in the presence of high concentrations of an irrelevant synthetic peptide corresponding to the N-terminus of the NDA4 molecule (K G N N D E S N I S F K E K D). TCL-ZL response to pp 22-35 at 1 AM (17×10³ cpm) was not significantly affected by the presence of NDA4-derived peptide at concentrations of 30-50 μM 15-17×10³ cpm), suggesting that high-zone tolerance was induced only by the specific immunogen. T cells cultured with 27 μM of pp 22-35 lost viability (>90% Preputium iodide positive) within 72 hours, while T cells grown with high concentration of the irrelevant peptide alone were unaffected

T Cell Reactivity of Transplant Patients to the Dominant Epitope of the DR1 Molecule

To determine whether both the direct and indirect pathway of allorecognition contributes to rejection, the T cell reactivity of three DR11 positive patients to DR1 positive APCs and to peptide 22-35, presented by self APCs were monitored at biweekly intervals for the first 3 months following transplantation and monthly thereafter. One of these patients, DT, was free of rejection for the entire period (12 months) of observation. IL-2 expanded T cells from the periphery failed to react to pp 22-35. None of the biopsies obtained from DT yielded growing lymphocytes.

The second patient, CB, was also free of acute rejection episodes during the first eleven months following transplantation, showing no lymphocyte growth from biopsies or histological evidence of cellular rejection. After 12 months, however, the patient entered chronic rejection developing anti-donor HLA antibodies and coronary artery disease. At this time, LDA analysis of IL-2 grown T cells from the periphery revealed the presence of Th capable of recognizing pp 22-35 in the presence of recipient APCs, at a frequency of 6×10⁻⁶ (Table 1). However, when tested for direct recognition of donor splenocytes, this IL-2 grown population of peripheral T cells failed to react in 3-day blastogenesis assays. Hence, the patient exhibited in the periphery, activated IL-2R positive T cells engaged in the direct but not in the direct pathway of alloreactivity.

TABLE 1
Frequency of Alloreactive T cells in the Circulation and Graft^{a}

T helper cell frequency in response to:

| | | Circulation | | | Graft | | |
|---|---|---|---|---|---|---|---|
| | | | DR1 | Donor | | DR1 | donor |
| | Time after | Self | pp22-35 + | Spleen | Self | pp22-35 + | spleen |
| Patients | Transplantation | PBMC | self PBMC | cells | PBMC | self PBMC | cells |
| DT | 12 Mo | 0 | 0 | 0 | No T cells | | |
| | | | | | grown out of | | |
| | | | | | biopsy | | |
| CB | 1 Mo | 0 | 0 | 0 | No T cells | | |
| | | | | | grown out of | | |
| | | | | | biopsy | | |
| | 12 Mo | 0 | 6.0 × 10⁻⁶ | 0 | No T cells | | |
| | | | | | grown out of | | |
| | | | | | biopsy | | |
| PI | 27 d | 0 | 0 | 0 | No T cells | | |
| | | | | | grown out of | | |
| | | | | | biopsy | | |
| | 41 d | 0 | 7.4 × 10⁻⁶ | 0 | No T cells | | |
| | | | | | grown out of | | |
| | | | | | biopsy | | |
| | 55 d | 0 | 8.0 × 10⁻⁶ | 0 | 0 | 4.3 × 10⁻⁴ | 1.1 × 10⁻² |
| | 82 d | 0 | 6.5 × 10⁻⁶ | 0 | 0 | 3.3 × 10⁻⁴ | 1.0 × 10⁻² |

^{a}Lymphocytes derived from Patients PBMC and graft were cultured in medium containing IL-2 for 14 days and then tested in LDA for reactivity for pp 22-35 and donor spleen cells, in a 3 day proliferation assay. The cell frequency was analyzed by Poisson statistics.

The third patient (PI) showed focal lymphocytic infiltrates in the grafts, consistent with acute rejection, and lymphocyte growth from the biopsy taken on day 55 and 82 following transplantation.

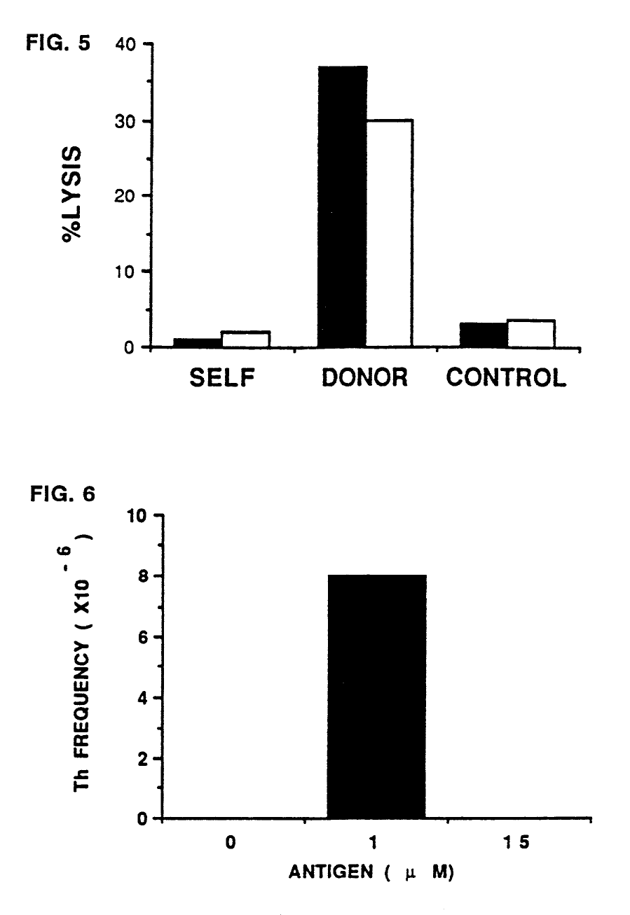

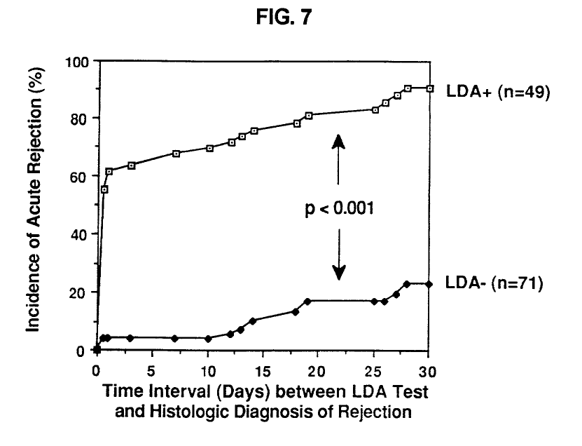

LDA analysis of peripheral blood T cells revealed the presence of pp 22-35 specific Th at a frequency of 7.4×10⁻⁶ as early as day 41, i.e. 14 days before acute rejection could be diagnosed by histology and lymphocyte growth assays (Table 1). This IL-2 expanded population of peripheral Th comprised no cells capable of direct recognition of donor APCs. Acute cellular rejection was documented histologically and by the lymphocyte growth assay on day 55 and 82. The frequency of Th reacting to pp-22-35 was 8×10⁻⁶ on day 55 and 6.5×10⁶ on day 82 in the periphery. These IL-2 expanded lymphocytes did not react against donor splenocytes. In contrast, lymphocyte cultures from day 55 and 82 biopsies showed frequencies of 1.1×10⁻² and 1×10² donor-reactive T cells, respectively. Day 55 and 82 cultures also contained T cells which recognized pp 22-35 of frequencies of 4.3×10⁻⁴ and 3.3×10⁻⁴ respectively (Table 1). Hence, at the time of rejection the graft was infiltrated by T cells recognizing donor alloantigens both via the direct and indirect pathway. However, the frequency of T cells capable of direct recognition was about 25-30 fold higher than that of T cells capable of indirect recognition. CML assays revealed the presence of cytotoxic T cells which produced 37% and 30% lysis of PHA-activated donor splenocytes on day 55 and 82, respectively (FIG. 5).

Cytofluorometric testing of T cells expanded from the biopsy showed that 80% of the cells had the CD4 and 20% of the CD8 phenotypes.

In vitro Inhibition of Patient's Reactivity to the Dominant Determinant of the DR1 Molecule. Because indirect T cell alloreactivity is limited to a single dominant determinant, selective immune interventions, such as induction of high zone tolerance may be useful to prevent rejection.

To establish whether the reactivity of in vivo activated T cells to peptide 22-35 can be suppressed by high concentrations of antigen the following experiment was performed: PBMC from patient PI were cultured for 14 days at different cell concentrations in medium with IL-2. Cultures were then tested in the presence of autologous APCs for reactivity to pp 22-35 at concentrations of 1 μM and 15 μM. At 1 μM of antigen there were 8×10⁻⁶ reactive T cells while at 15 μM there was no reactivity (FIG. 6). These data indicate that high concentrations pf allopeptide can inhibit the reactivity of T cells engaged in the indirect pathway.

Discussion

The role of direct recognition in allograft rejection has been extensively documented (Sherman and Chattopadhyan, 1993; and Shoskes and Wood, 1994). Significantly less is known, however about the role of indirect recognition.

Although the indirect recognition pathway is not expected to generate a very high precursor frequency of alloreactive T cells, such helper T cells may play an important role in initiating and perpetuating chronic rejection. Thus, T helper cells recognizing allopeptides derived from the processing of donor MHC antigens may produce lymphokines which mediate antibody production, Tc activation, DTH-type reactions, endothelial cell injury and subsequent arteriosclerotic lesions.

The aim of the present study was to determine whether indirect recognition occurs in transplant recipients undergoing acute of chronic rejection. To follow the activation of the indirect T cell recognition pathway in transplant recipients the dominant immunogenic peptide of the DR1 molecule was first identified. T cells immunized in vitro with pp 21-42 reacted to soluble rDR1 protein, indicating that the dominant epitope of the DR1 molecule resides within these residues. Further epitope mapping experiments showed that the core of this dominant determinant corresponds to residues 22-35. A cryptic determinant residing within residue 1-20 was also found. Although, the possibility that determinant spreading to cryptic determinants may occur (Sercarz et al., 1993) during chronic antigenic stimulation cannot be dismissed, the fact that both in man and in mice (Benichou et al., 1994) a sole determinant appears to from the dominant T cell epitope, opens the way to specific immunotherapy.

Having identified pp 22-35 as the dominant determinant of the DR1 molecule, whether this peptide is recognized by activated T cells from DR11 positive patients transplanted with a DR1 positive graft was determined. Using an experimental approach similar to that described by Zhang et al. for the study of T cells specific for MBP (Zhang et al., 1994), the ability of activated, IL-2 responsive T cells from the peripheral blood and from graft tissue to react directly or indirectly against the DR1 antigen was tested.

In previous studies it was showed that Th cells capable of recognizing an allo-MHC peptide presented by self APC, are present in the circulation of healthy individuals at a frequency of about 1/250,000 (Liu et al., 1992). The corresponding frequency of MLC-reactive Th cells, capable of direct recognition, is at least 100 fold higher (Liu et al., 1993). However, in healthy individuals these cells are present in the circulation in a resting state and become activated only after in vitro immunization with allogeneic cells or MHC allopeptides. IL-2 expanded lymphocytes from the blood of healthy individuals or patients transplanted with a DR1-negative graft, show no direct or indirect reactivity against the DR1 antigen.

Analysis of IL-2 expanded PBMCs from DR11 positive recipients transplanted with a DR1 positive heart, showed that neither pp 22-35 or DR1 positive APCs elicited T cell reactivity, while the patients were in quiescence. However, prior to and during rejection, pp 22-35-reactive-T cells were present in the circulation suggesting that indirect recognition of donor alloantigens takes place in the periphery.

The IL-2 expanded population, however, comprised no cells reacting directly against donor splenocyted indicating that T cells activated by direct recognition of graft HLA Class II antigens are absent from the peripheral blood T cells from patients undergoing rejection do not display accelerated responses against donor APC in 3-day MLC.

Analysis of T cells infiltrating the graft at the time of rejection revealed a heterogeneous population consisting of Th cells reacting with peptide 22-35 presented by recipient APC, Th cells reacting with the DR1 antigen expressed by donor APCs and Tc cells capable to lyse specifically donor cells. Study of Th frequencies showed that direct recognition prevailed over indirect recognition within the population of lymphocytes infiltrating the graft. This observation requires some explanation, however, because it is assumed that direct recognition of the allograft By CD4⁺ helper T cells relies heavily on the presence of MHC-Class II positive donor APCs in the grafter tissue. Since such cells migrate out of the graft relatively early following transplantation, it is expected that the role of direct T cell recognition of the graft is confined to early acute rejection (Sherman and Chattopadhyan, 1993; Shoskes and Wood, 1994; and Lechler and Batchelor, 1982). Because the biopsies, from which T cells were grown, were obtained on day 55 and 82, it is possible that they still contained donor MHC-Class II positive APCs.

On the other hand, if donor APCs had already left the graft, direct recognition may have been triggered by vascular endothelial cells expressing high levels of HLA-DR antigens. Upregulation of MHC-Class II expression might have been induced by gamma interferon secreted by Th involved in indirect recognition. This possibility is supported by the finding that pp 22-35 reactive T cells were already present in the periphery two weeks prior to rejection. Hence, indirect T cell recognition, may actually take place before direct recognition, due to the processing and presentation by host APCs of MHC-Class II antigens shed from the graft. The subsequent activation of the direct T cell recognition pathway should result in the expansion of a wide repertoire of alloreactive T cells, capable to recognize the many different peptides bound to the donor MHC molecules. This population is likely to overgrow the oligoclonal population of T cells recognizing indirectly the dominant determinant of the allogeneic MHC molecule. CD4 positive T cells, activated directly or indirectly by donor MHC molecules, may provide help to graft specific CD8⁺ cytolytic T cells. Because the recipient antigen-presenting cells are always available, T cell recognition of precessed forms of donor's MHC allo-antigens may take place at any time, contributing to antibody production and chronic rejection.

The fact that the fine specificity of indirect allorecognition is limited to a single determinant of donor MHC molecule, and that there is a limited usage of TCR Vβ genes by T cells responding to allopeptides (Liu et al., 1993) suggests that selective immunointervention could be achieved by TCR vaccination, treatment with anti-TCR antibodies or tolerance induction to the dominant epitope, as proposed for autoimmune diseases (Critchfield et al., 1994; Howell et al., 1989; Vandenbark et al., 1989; and Krensky and Clayberger, 1995).

In an effort to explore the possibility that the response to the dominant epitope can be abolished the effect of high concentrations of peptide 22-35 on the reactivity of in vitro and in vivo activated T cells was investigated. It was found that the response of allopeptide specific T cells, which have been activated in vitro and in vivo, is suppressed by high concentrations of peptide 22-35. Induction of high zone tolerance may therefore, be a viable option for preventing rejection.

This is the first demonstration that activated T cells involved in the indirect pathway of allorecognition are present at the site of allograft rejection as well as the periphery and that their reactivity can be specifically blocked by high concentrations of antigen.

### EXAMPLE 2

### Indirect Recognition Pathway Suppressed by TCR Antagonist Based on the Dominant HLA-DR Epitope

Acute rejection is mediated primarily by T cells engaged in the direct recognition pathway which involves both the allogeneic MHC molecule and its associated peptide ligand. Because of molecular mimicry with self MHC-peptide complexes, the allogeneic target (MHC plus peptides) elicits a very strong and heterogeneous response, in which as many as 10% of T cells can be engaged. As expected the T cell receptor repertoire specific for alloantigen is diverse. In spite of its sudden and violent character, acute rejection can be reversed by use of steroids, OKT3, ATC and increased doses of conventional immunosuppression. However, it has been shown that during acute rejection soluble alloantigen is released from the injured graft and stimulates anti-HLA antibody production (Lechler et al., 1990 and Lo et al., 1989). The antigen must, therefore, processed by APCs and presented in the form of MHC-bound allopeptides to T cells, resulting in systemic activation of both the cellular and humoral arm of the alloimmune response. The hypothesis is that allopeptide reactive T cells provide the helper factors required for the production of anti-HLA antibodies which mediate graft atherosclerosis and chronic rejection.

To prevent chronic rejection the indirect recognition pathway should be blocked in a specific way which does not compromise immune homeostasis. This implies that T cells recognizing the dominant epitope should be “tolerized”, e.g., energized or deleted.

One of the most efficient approaches for specific immunosuppression resides in the use of TCR antagonists. Such antagonists can be generated by changing crucial T cell contract residues on peptide antigens. Complexes between MHC and TCR antagonists bind to the same region of the TCR normally engaged by antigen/MHC complexes, and block T cell reactivity.

Design of TCR Antagonists

The identification of the core epitope of the DR4/69-88 peptide has been accomplished. From that information, the peptide spanning residues 71-84 was deemed the most potent in stimulating the proliferative response of TCL-PR. The peptide KRAAVDTYCRHNYG carries the expected anchor residues required by the peptide binding motif of the DTRβ1*1101 restriction molecule (i+7) as well as the TCR contact residues involved in the stimulation of TCL-PR.

| | |
|---|---|
| | TCR |
| Peptide 4/71-84 | K R A A V D T Y C R H N Y G (Seq. I.D. No. 11) |
| | MHC |
| Peptide binding motif | |
| of DTRβ1*1101 (i + 7) | Hy,X,X,X,X,X,X,R/K |

The methodology for the design and analysis of hemagglutinin TCR antagonists have been previously described by A. Sette et al., and may be adapted to obtain synthetic peptides which bind efficiently to HLA-DTRβ1*1101 yet fail to stimulate antigen specific proliferative responses in TCL-PR.

The panel of synthetic analogues of 4/71-84 which may be synthesized will retain the alanine and arginine anchor residues shown to be important in DR11 binding. The secondary anchors, within the sequence, probably reside at i+3 and i+5. Thus, the probable TCR contact residues are in positions i−1, i+l, i+2, i+4, i+6 and perhaps i+8. First, the focus is on preparing analogs containing substitutions at positions i+1, i+2, i+4, and i+6. The substitutions which have been most successful at producing powerful antagonists have been isosteric.

| | | |
|---|---|---|
| KRAGVDTYCRHNYG (Seq. I.D. No. 12) | 4/71-84 | 74 A to G |
| KRAVVDTYCRHNYG (Seq. I.D. No. 13) | 4/71-84 | 74 A to G |
| KRASVDTYCRHNYG (Seq. I.D. No. 14) | 4/71-84 | 74 A to S |
| KRAAADTYCRHNYG (B1) | 4/71-84 | 75 V to A |
| KRAAGDTYCRHNYG (B2) | 4/71-84 | 75 V to G |
| KRAVLDTYCRHNYG (B3) | 4/71-84 | 75 V to L |
| KRAAVDSYCRHNYG (B4) | 4/71-84 | 77 T to S |
| KRAAVDDYCRHNYG (B5) | 4/71-84 | 77 T to D |
| KRAAVDVYCRHNYG (B6) | 4/71-84 | 77 T to V |
| KRAAVDTYSRHNYG (B7) | 4/71-84 | 79 C to S |
| KRAAVDTYARHNYG (B8) | 4/71-84 | 79 C to A |
| KRAAVDTYNRHNYG (B9) | 4/71-84 | 79 C to N |

To increase resistance against enzymatic degradation the first residue is synthesized with D lysine rather than with L lysine. The terminal carboxyl group is also amidated.

Blastogenesis and Binding Assays for Testing of Antagonistic Peptides

This panel of analogs will be first tested for their ability to stimulate the proliferation of TCL-PR at doses ranging from 1 to 10000 nM, using the parent peptide 4/71-84 as a control. The relative antigenicity will be expressed as the dose required to produce the half-maximal proliferation of TCL-PR. From this experiment it was possible to determine the relative antigenicity of each peptide and identify peptides which may have partially or completely lost their ability to stimulate TCL-PR. It is possible that some these peptides will retain their ability to stimulate TCL-PR and thus, should be classified as agonists rather than competitive antagonists. The lack of antigenicity of antagonist peptides to PBMCs from Dr⊕1*1101 positive responders will be confirmed in LDA experiments.

Next, the relative capacity of each analogs to act as a TCR antagonists may be assessed. APC may be prepulsed for 2 hours with a suboptimal dose of 4/71-84 peptide or recombinant DR4, followed by washing to remove unbound Ag. APC were then incubated with various concentrations of the analogs. Since very low levels of Ag occupancy of MHC molecules on the APC surface are required to achieve a suboptimal T cell response, and since the half-life of MHC-AG complexes is long, the APC surface will present a fixed amount of antigen and varying amounts of MHC-analog complexes. This permits the assessment of TCR antagonism. To avoid possible complications due to recycling of MHC and/or back signaling from the APC to the T cells, APCs may be pulsed and then fixed. By comparing the antigenicity and antagonism data, the analogs may be placed into one of the following four categories: 1) Agonist peptides which retain some degree of antigenicity 2) Antagonist peptides which demonstrate pure antagonistic behavior in the absence of any antigenicity. 3) Partial agonist/antagonist which elicit lymphokine production or display some antigenicity at high doses and some antagonism at lower nonantigenic doses. 4) Null peptides that demonstrate neither antigenicity nor antagonism.

In parallel experiments one may measure the binding affinity of each analogue to the HLA-DRβ1*1101 molecule relative to the parent peptide. For these experiments one may employ the system which was previously described. Briefly, soluble Drβ1*1101 molecules (5-10 nM) purified from SF9 insect cells are incubated in the presence of 5 nM ¹²⁵I radiolabeled peptide 4/71-84 and various concentrations (1 to 10000 nM) of analogue peptide, in protease inhibitor cocktail, for 48 hours at 26° C. The DR peptide complexes are separated from free peptide by high performance gel filtration chromatography on a TSK column. Fast eluting counts represent the DR-peptide complexes. These counts are plotted versus the concentration of analogue used in the inhibition assay to obtain the dose at which 50% inhibition of binding of peptide 4/71-84, yet fail to stimulate TCL-PR, act by antagonism rather than MHC competition or blockade (because competition can occur only if the binding affinity is significantly higher).

Results

From these series of experiments one may identify one or more competitive, antagonists peptides which bind to Drβ1*1101 in the nM range yet fail to stimulate antigen specific proliferation of TCL-PR. Such peptides should bind well in the groove of Drβ1*1101, engage the TCR expressed by TCL-PR, yet they should fail to initiate successfully the signaling pathway which leads to T cell proliferation. The capacity of the analog peptides to inhibit T cell responses in this manner will be measured at the subcellular, cellular and intracellular levels.

Antagonist peptides which eliminate indirect recognition of allopeptides processed and presented by recipient APC have the potential of becoming therapeutic tools for the treatment of chronic rejection.

Possible Problems and Alternative Approaches

It is possible that using the initial panel of 12 analog peptides one may fail to identify a DR4 specific antagonist. These 12 analog peptides (4 sets of 3) may have been generated by performing substitutions at positions i+1, i+2, i+4 and i+6 (three substitutions at each positions). In the event that it is not successful one may try other amino acids for substitutions at these positions and at the secondary anchors (i+3 and i+5).

T Cell Unresponsiveness to the Dominant Allopeptide Epitope Induced by High Dose Antigen. TCR Agonists or MHC-peptide Complexes

MHC-associated self peptides mediate both positive and negative selection. One explanation for this conceptual paradox has led to the following affinity-avidity model. In the thymus, TCRs of the immature thymocyte encounter MHCs loaded with a variety of peptides that have different binding affinities to the TCR and are expressed at different densities on the surface of selecting cells. A relatively high affinity TCR-peptide/MHC interaction can lead to positive selection as long as the density of the TCR and that of the peptide-MHC complex are below certain levels. Cell undergoing negative selection (tolerance) may receive more of the same signals than that received by cells undergoing positive selection. Alternatively, engaged TCRs at high densities may generate signals that are qualitatively different from the signals generated by engaged TCRs at low densities. Since TCR agonists represents structural variants of the dominant epitope which maintain antigenicity, yet can be selected for higher affinity for MHC, they can be useful for inducing tolerance at relatively lower doses.

Another way of inducing, tolerance consists of using the peptide antigen complexed to soluble MHC of responder type. Antigenic presentation occurs, in this case, in the absence of costimulatory molecules which stimulate T cell activation.

These three related strategies may be explored for tolerance induction (high dose tolerance, TCR agonists, and peptide-MHC complexes) using as a tool TCL-PR, which is specific for the dominant epitope of DR4 presented by Drβ1*1101. The nature of the biochemical changes which underlie the loss of the T cell proliferative ability will be analyzed at the level of signal transmission and lymphokine transcription.

Induction of High Dose Tolerance

The objective of these studies is to establish whether high doses of dominant peptide are tolerogenic. To increase resistance against enzymatic degradation D-Lysine is placed at the N-terminus and the C-terminus is amidated. We have established that the optimal dose of the dominant peptide (4/71-84) required for inducing maximal proliferation of TCR-PR is 5 μg/ml or approximately 5×10⁻⁷M. Increasing concentration of peptide 4/71-84 (ranging from 10⁻⁸ to 10⁻⁴M) may be used to determine the dose required to reduce proliferation to the background values observed in cultures with APCs and no peptide. The viability of the cells will be assayed and physiological studies will be performed to determine whether the lack of proliferation results from the induction of enery of apoptosis.

The work that may be done to examine apoptotic cell death include preputium-iodide staining-DNA-laddering and FACS analysis using biotin labeled nucleotides and TdT transferase an indicator of DNA fragmentation are described below. For the DNA laddering assays T cells (1×10⁶) are pelleted and lysed in buffer containing 0.09M Tris, 0.09M boric acid, 0.002M EDTA, 75 μg/ml proteinase K, 2% SDS and 50 u/ml of Rnase and incubated on ice for 2 hours. The cell extracts are diluted in gel loading buffer prior to agarose gel electrophoresis. DNA is then visualized by a UV transilluminator.

The FACS procedure to show DNA breaks is as follows: 5×10⁵ cells are fixed in 1% formaldehyde, centrifuged and resuspended in ice cold 70% ETOH to permeabilize the cell membranes. To link the DNA breaks to Biotin®, the cells are mixed in a reaction mixture containing 37.8 ml of MilliQ® water, 5 ml TdT buffer (1M sodium cacodylate, 1 mM DTT, 0.5 mg/ml of serum albumin, 5 ml of CaCl2 (25 mM), 2 ml of Biotinylated® 11-dUTP and 0.2 ml of TdT enzyme. The total mixture is incubated for 30 minutes at 30° C., washed and resuspended in PBS. The cells are then stained with staining buffer containing 25 ml of SSC (20×), 54 ml of MilliQ® water, and 0.7 ml of Avidin® FITC. The cells are incubated at room temperature in the dark, washed and analyzed by FACS.

If unresponsiveness is not determined by apoptosis, then one may investigate whether reactivity can be restored by adding IL-2. Endogenous production of IL-2 and IL-4, following high dose tolerization, may be measured by quantitative PCR. If unresponsiveness is determined by apoptosis one may rule out the possibility that high doses of the tolerogen are toxic, using high doses of an identically prepared peptide which binds to HLA-Drβ1*1101 but does not stimulate TCL-PR. DR1 which bind to Drβ1*1201 may be used as a control.

Induction of Tolerance by Use of TCR Agonists

The methodology for the design of TCR agonists has been previously described. A panel of 4/71-84 analogs containing the following substitutions at probable DR contact positions may be prepared. Anchor residues reside at i and i+7. Secondary anchors may reside at i+3 and i+5. Agonists may be prepared as shown below.

Parent Peptide 4/71-84 KRA(i)AVD(i+3)TY(i+5)CR(i+7)HNYG**

Possible agonist analogs:

```
Parent Peptide 4/71-84 KRA (i) AVD (i + 3) TY (i + 5) CR (i + 7) HNYG**
Possible agonist analogs:
          4/71-84 73 A→Y  Y
                        73 A→L  L
                        73 A→I  I
                        76 D→E  E
                        78 Y→F  F
*D-Lysine  **Amidated COOH terminus
```

First one may perform a comparison of agonists binding to recombinant HLA-DR1101 protein using as a standard the parent peptide. These experiments may identify the analog(s) which have the highest binding affinity and are likely to display higher MHC occupancy than the parent peptide.

Next it may be established whether the agonists is still antigenic and stimulates the proliferation of TCL-PR. If this is the case then the dose may be determined at which this agonists may allow one to identify peptides which are more potent than the parent peptide for induction of tolerance. Again, functional test may be performed to characterize possible changes undergone by TCL-PR as a consequence of tolerance induction.

Induction of Tolerance by Use of Soluble MHC/peptide Complexes: in vitro Models

Soluble MHC/peptide complexes were previously shown to be efficient for antigen-specific therapy of EAE. Soluble HLA-DRβ1*1101 molecules will be purified form SF9 transfectants using the methodology detailed in previous studies. Purified Drβ1*1101 protein can be loaded with peptide using a 0.50 fold molar excess of peptide relative to DR in a 48 hour incubation at 37° C. Previous investigations have shown that 20% occupancy can be achieved. In some preliminary experiments it was found that loading was more efficient in slightly acidic conditions, which induce a conformational change in the structure of HLA-Dr, that is more favorable to peptide exchange. Under these conditions it can be obtained up to 40% occupancy but with lower yield, as some of the HLA-DR molecules denature. MHC/peptide complexes are purified by gel filtration/HPLC as previously described.

The dominant peptide epitope 4/71-84 will be used for loading the Dr 1101 protein. In a first series of experiments. TCL-PR (×10⁶) will be cultured with 1-10 g of HLA-DR 1101 protein loaded with peptide 4/71-84, a control peptide or with medium alone. After 24 hours the cells will be washed and then cultured with fresh APCs, peptide 4/71-84 at the optimal concentration (5 ug/ml) or recombinant iL-2 (20 units per ml). ³H-TdR incorporation will be measured after 72 hours. Treatment with DR 1101-peptide 4/71-84 complexes may result in inhibition of clonal proliferation.

It is possible, however, that inhibition can also be obtained using complexes of MHC protein with agonists which display altered avidity for the TCR. Agonist peptides may be loaded to MHC protein at the same level of occupancy as the achieved by peptide 4/71-84. The two types of MHC-peptide complexes, e.g. one with 4/71-84 and the other with the analog, may be compared in a dose-response titration experiment to establish their relative effectiveness as tolerogens.

Biochemical Characterization of Signaling Events in Anergized T Cell Clones

Anergized T cells are defective in their ability to release intracellurlar calcium and are unable to synthesize IL-2, IL-4, IFN mRNA or secrete measurable IL-2 after antigenic stimulation. This hyporesponsiveness is reversible if the cells are stimulated with PMA and ionomycin. To determine whether any of the methods which are planned to be used for tolerance induction renders the cells anergic by these subcellular criteria, the measurement of Pl metabolism, Ca+2 and cytokine transcription after suppression to TCL-PR reactivity may be carried out.

An early biochemical event associated with TCR activation in T cells is the release of soluble inositol phosphates (InsP1-5)from the membrane phosphoinositide pool. The measurement of inositol phosphates is readily performed with small numbers of T cells using a modification of a previously described technique. Briefly, the tolerized T cell clones are labeled with ³H inositol. DRB1*1101 positive APC are loaded with parent peptide 4/71-84 and incubated overnight. Labeled T cells are added to each well and TCR activation is allowed up to 10 min. Incubation is terminated by the addition of acid. The acid extract is neutralized and applied to a Whatman Partisil SAX® 10 column equilibrated in water. The InsP1, InsP2 and InsP3-5 metabolites are eluted in a step gradient of ammonium phosphate. Authentic InsP standards are used to calibrate the column. By this method it may be possible to compare the phospoinositide hydrolysis resulting from T cell activation initiated by the ligand (i.e. parent 4/71-84 peptide/MHC complex).

If there is no increase in soluble inositol phosphate metabolites (InsP3) it may be concluded that the tolerogen has interfered with PLC activation and the hydrolysis of phosphoinosides.

Peptide-reactive T cell clones release Ca++ from their intracellular storage pool into the cytosol in response to the specific antigen presented by MHC. Anergized T cells have a marked reduction in their release of Ca++ in response to the specific antigen, although their response to ionomycin remains unchanged.

“Anergized” T cells will be loaded with Indo-1 in culture media and analyzed by fluorescence. Next, stimulator APCs, which have been pulsed with 4.0 uM 4/71-84 peptide may be added, and the flow analysis is repeated. Unpulsed APCs are used as controls. The mixture of “anergized” T cells and APCs will be centrifuged to establish cell-cell contact, and then resuspended and analyzed for response. The fluorescence shift should be diminished if bona fide anergy has occurred. Ionomycin (100 mg/ml) is used as a positive control for Indo-1 loading.

Detection of Cytokine mRNA by Northern Analysis

It is known from in vitro experiments that in response to antigen, certain anergic clones produce lymphokines such as lFNg, lL3, GMCSF in the absence of lL-2 production. Similarly in vivo anergized T cells, which proliferate poorly in response to antigen, may retain some normal lymphokine response.

One embodiment of this invention is to ablate not only the proliferative capacity of T cells but also lymphokine production. One may monitor by Northern Blot Analysis the transcription of IL-2, IL4, IFNy, TNFB, IL-3 and GMCSF.

### EXAMPLE 3

### Blockage of Allospecific T-B Interaction in ex-vivo and SCID-mouse Models

The cooperation of allopeptide specific T helper cells with B cells with Ig receptors for the same alloantigen results in the production of anti-HLA antibodies which cause chronic rejection. The following scenario is envisioned: B cells bind antigen with their antigen receptor membrane immunoglobulin (Ig) and then internalize and present processed peptides on their MHC-class II molecules to T cells. Allopeptide-specific T cells recognize the processed antigen on the B cell surface, become activated, and drive B cell maturation. Chronic rejection is accompanied by the release of in the circulation of donor HLA antigens and the production of anti-HLA antibodies. T cells which are able to recognize peptides derived from the processing of allogeneic MHC molecules are able to provide antigen-specific “help” to autologous B cells.

Enumeration of Anti-DR. Ig Secreting B Lymphocytes in DR1101 Positive Recipients of a DR. Transplant

These studies establish the frequency of anti-DR. Ig producing B cells in patients' peripheral blood circulation, using the ELISA Spot or RIA assays described hereinabove. To compare the number of Ig-secreting B lymphocytes among samples obtained before and during rejection, the values may be normalized to the number of Ig-secreting B cells/105 PBMC. Sequential bleedings are cryopreserved from 12 DR11 positive recipients of a DR. allograft.

Quantitation of Allospecific B Cells after in vitro Stimulation

T and B cells will be purified from PBMCs obtained at the time of transplantation and later when anti-DR. antibodies were first seen in the patients' serum. B cells will be stimulated with 1) rDR4, 2)rHLA-DR absent in the donor (negative control) and 3) tetanus toxoid (positive control). Specific Inhibition of T helper Activity. Specific Inhibition of Th-B cell cooperation in SCID mice transplanted with patients' PBMC.

### EXAMPLE 4

### Indirect Recognition of DonorHLA-DR Peptides in Organ Allograft Rejection

To determine whether indirect allorecognition is involved in heart allograft rejection T cells obtained from peripheral blood and graft biopsy tissues were expanded in the presence of IL-2 and tested in limiting dilution analysis (LDA) for reactivity to synthetic peptides corresponding to the hypervariable regions of the mismatched HLA-DR antigen(s) of the donor. Serial studies of 32 patients showed that T cell reactivity to donor allopeptides was strongly associated with episodes of acute rejection. The frequency of allopeptide reactive T cells was 10-50 fold higher in the graft than in the periphery indicating that T cells activated via the indirect allorecognition pathway participate actively in acute allograft rejection. In recipients carrying a graft differing by two HLA-DR alleles the response appeared to target only one of the mismatched antigens of the donor. Indirect allorecognition was restricted by a single HLA-DR antigen of the host and directed against one immunodominant peptide of donor HLA-DR protein. However, intermolecular spreading was demonstrated in patients with multiple rejection episodes by showing that they develop allopeptide reactivity against the second HLA-DR antigen. These data imply that early treatment to suppress T cell responses through the indirect pathway of allorecognition, such as tolerance induction to the dominant donor determinant, may be required to prevent amplification and perpetuation of the rejection process.

Introduction

The long-term function of organ allografts is often compromised by rejection, a complex phenomenon which involves both the cellular and the humoral arms of the immune response. The mechanism of allograft rejection is not completely understood, although it has become increasingly clear that recipient T helper (Th) cells become activated upon direct recognition of MHC/peptide complexes present on the membrane of passenger dendritic cells of donor origins (Steiman and Witmer, 1978; Mason et al., 1981; Lechler and Batchelor, 1982; and Matzinger and Bevan, 1977). This vigorous response, which appears to violate the rule of self MHC restriction, is driven primarily by antigenic mimicry (Lechler et al., 1990 and Lechler et al., 1992). In non-sensitized transplant recipients, T cells activated via the direct allorecognition pathway are thought to be important for initiation of early acute rejection. However, these directly activated T cells seem to be less critical at later times, following departure of donor dendritic cells from the graft, because upon recognition of donor MHC molecules on “non-professional” antigen presenting cells (APCs) that lack co-stimulatory elements, they may become anergized (Lo et al., 1989; Gaspari et al., 1988; Bradley, 1996; and Benichou and Fedoseyeva, 1996).

More recently, several lines of evidence have indicated that the indirect recognition pathway, which describes the stimulation of recipient T cells by allopeptides processed and presented on self-MHC expressed by recipient APCs, is a major contributor to rejection. Thus, during rejection of skin, kidney of heart allografts, T cells recognizing processed forms of donor MHC antigens associated with host restriction elements were detected in the primary lymphoid organs of mice and rat recipients (Benichou et al., 1992; Watschinger et al., 1994; and Sayegh and Carpenter, 1996).

Animals immunized with soluble class I or class II allogeneic MHC molecules or with synthetic MHC allopeptides were capable of accelerated rejection of skin allografts (Dalchau et al., 1992; Fangmann et al., 1992; and Sayegh et al., 1992). Studies using MHC class II deficient mice as donors showed that in the absence of direct recognition T cells activated via the indirect pathway initiate rapid skin allograft rejection (Auchincloss et al., 1993 and Lee et al., 1994). Alloreactive, self MHC restricted T cells were shown to provide the lymphokines required for the generation of cytotoxic T lymphocytes (CTL), alloantibodies and delayed-type hypersensitivity (DTH) responses (Bradley, 1996; Fangmann et al., 1992; Auchincloss et al., 1993; Lee et al., 1994; and Parker, 1993).

In the human model it has been demonstrated that two distinct populations of T cells, one involved in the direct and the other in the indirect recognition pathway, are activated after mixed lymphocyte culture (MLC) stimulation (Liu et al., 1993). Furthermore, T cells immunized in vitro with synthetic allopeptides respond specifically to the native alloantigen in the presence of self-APCs (Liu et al., 1992; Liu et al., 1995; and Colovai et al., 1996).

Both in mice and in humans, T cell proliferation to an allogeneic MHC protein was limited to a single dominant determinant and restricted by a self-HLA-DR molecule (Benichou and Fedoseyeva, 1996; Liu et al., 1992; Colovai et al., 1996; and Benichou et al., 1994). T cell receptor (TCR)-Vβ gene usage by T cells responding to the dominant allopeptide was also shown to be limited (Liu et al., 1992; and Liu et al., 1993). This suggests that indirect recognition is amenable to specific immunointervention (Bradley, 1996; Benichou and Fedoseyeva, 1996; Sayegh and Carpenter, 1996; and Colovai et al., 1996).

A detailed understanding of when and how the indirect pathway becomes activated in immunosuppressed organ allograft recipients should provide valuable insight into the development of strategies to block T cell alloreactivity through the indirect pathway.

Because allopeptide-specific T cells require that donor alloantigens be shed from the graft, proteolyzed and the resulting fragments bound to a MHC class II molecule of the host, it is important to establish whether their activation is the cause or consequence of early acute rejection and whether they recognize single or multiple determinants of donor HLA-DR antigens. To address these questions, we have evaluated the frequency of activated T cells recognizing donor allopeptides has been evaluated, in peripheral blood and biopsy tissue of heart allograft recipients during the early post-transplantation period. In vivo activated T cells were expanded in IL-2 and tested in LDA for reactivity to synthetic peptides corresponding to each of the mismatched HLA-DR antigens of the donor. The results provide evidence to support the role of the indirect recognition pathway in recognition. The data also suggest that one of the mismatched HLA-DR antigens is preferentially targeted by alloreactive T cells. Selective immunosuppression by be required for ablation of the immune response to this specific determinant.

Materials and Methods

Patient Specimens

Thirty-two heart recipients were recruited for these studies during the first 10 weeks following transplantation. All gave informed consent under the auspices of the appropriate Institution Review Board. Patients were treated with cyclosporin A, prednisone and azathioprine. Endomyocardial biopsies (EMB) were performed routinely for monitoring rejection according to a standard time schedule (Fisher et al., 1995). Four biopsy fragments were processed for histologic analysis and one was made available for immunologic studies (Fisher et al., 1995). Blood specimens were obtained from the patients at the time of EMB. Acute rejection, diagnosed on biopsies as histologic grade 1B, 2 or 3A according to Billingham's criteria (Billingham, 1995), was treated with increased doses of immunosuppression.

HLA Typing

All recipients and donors were typed for HLA-DR antigens by conventional serology and by molecular methodology using PCR-SSOP (sequence specific-oligonucleotide probe).

Peptide Synthesis

Synthetic peptide corresponding to residues 1-19, 21-39, 62-80 of DRβ1 chain from 32 HLA-DR alleles (DRβ1*0101, DRβ1*0102, DRβ1*0301, DRβ1*0302, DRβ1*0401, DRβ1*0402, DRβ1*0403, DRβ1*0404, DRβ1*0405, DRβ1*0407, DRβ1*0408, DRβ1*0701, DRβ1*0801, DRβ1*0802, DRβ1*0803, DRβ1*0804, DRβ1*0901, DRβ1*1001, DRβ1*1101, DRβ1*1102, DRβ1*1104, DRβ1*1201, DRβ1*1301, DRβ1*1302, DRβ1*1303, DRβ1*1401, DRβ1*1402, DRβ1*1501, DRβ1*1502, DRβ1*1503, DRβ1*1601, and Drβ1*1602)(Bodmer et al., 1994) were obtained from Chiron Mimotopes (San Diego, Calif.) and Peptide Innovation (Raleigh, N.C.). The purity of peptides was higher than 90%, as indicated by HPLC and mass spectometry. Synthetic peptides were dissolved in RPMI 1640 medium at a concentration of 2 mg/ml.

Limiting Dilution Analysis of Allopeptide Reactive T Cells in the Peripheral Blood

PBMCs were grown for 7 days in 96 well trays at concentrations of 4×10⁴, 2×10⁴, 1×10⁴ per well (24 wells for each concentration) in RPMI 1640 medium supplemented with 10% human serum (Sigma, St. Louis, Mo.) and 50 units recombinant (r)IL-2/ml (Boehringer Mannheim, Indianapolis, Ind.), as described by Zhang et al. 1994. On day 7, plates were washed three times and each culture was split into four aliquots. Two plates were used for testing T cell reactivity to a cocktail of peptides (1 μAM of each peptide) corresponding to each of the mismatched HLA-DR antigens of the donor in the presence of irradiated autologous PBMCs (5×10⁴/well). The third plate to which only self PBMCs (without peptides) were added, served as a negative control. After 48 hours of incubation, the cultures were labeled with [³H]TdR and harvested 18 hours later. The forth plate was left unlabeled and kept for T cell expansion and HLA-DR restriction studies. The frequency was calculated as previously described (Zhang et al., 1994).

Limiting Dilution Analysis of Allopeptide Reactive T Cells Infiltrating the Graft

Graft tissue (1 mm³/piece) was placed in 24 well plates containing 1×10⁶ autologous APCs (irradiated with 3000 rad) in RPMI 1640 medium supplemented with 5 units rIL-2/ml and 10% human serum. After 7 days the LDA was set up as described above. The cell concentrations were 800, 400 and 200 cells/well for 24 replicate reactions. Fifty-thousand autologous APCs were added to each well. On day 7 the wells were split as above and T cell reactivity to allopeptides was tested in a 3 day blastogenesis assay.

Analysis of Dominant Allopeptide Determinants and of Restriction Elements

Cells from cultures corresponding to wells in which T cell reactivity to donor allopeptides was detected were expanded in 24 well plates in the presence of autologous APCs (1×10⁶/well), stimulating peptide mixture and rIL-2 (20 u/ml). Cultures were restimulated every 14 days. After 3 or 4 stimulations the resulting T cell lines were tested for reactivity to the individual peptides comprised in the mixture, in the presence of APCs from the recipient and from hemi-allogeneic donors sharing one HLA-DR antigen with the recipient. Allopeptide-reactivity was measured in 3 day blastogenesis assays as previously described (Liu et al., 1992).

Statistics

Statistical analyses were performed using BMDP statistical software (BMDP Statistical Software, Inc., Los Angeles, CA). The incidence of rejection one month following LDA was calculated by the product-limit method. Differences between groups were compared using Breslow statistics. A test of linear trend was used to analyze the relationship between allopeptide reactivity and progression of acute rejection (Dixon et al., 1990).

RESULTS

Activation of the Indirect Recognition Pathway During

Allograft Rejection

To determine whether indirect allorecognition contributes to allograft rejection, T cells obtained from the peripheral blood of 32 heart transplant recipients were expanded in medium supplemented with rIL-2 and then tested for reactivity to synthetic allopeptides corresponding to the hypervariable regions of the mismatched HLA-DR antigens of the donor. Characteristics of the study population are shown in Table 2.

TABLE 2
Characteristics of Study Population

| | No. of Patients |
|---|---|
| Total | 32 |
| Gender | |
| Male | 28 |
| Female | 4 |
| Mismatched for | |
| 1 HLA-DR | 13 |
| 2 HLA-DR | 19 |
| LDA initiated on | |
| 1st-2nd week | 12 |
| 3rd-4th week | 11 |
| 5th-10th week | 9 |

A minimum of two serial PBMC specimens (mean 3.5) were obtained from each patient with elapsed intervals of 1 to 12 weeks (mean 4.1 weeks) between collections. Following 7 days of expansion in medium containing rIL-2, T cells were tested in LDA for reactivity against synthetic peptides corresponding to each HLA-DR antigen of the donor. T cell proliferation in response to one or both sets of donor DR peptides was considered indicative of in vivo activation of the indirect recognition pathway. Forty-nine out of the 120 samples of PBMCs studied in LDA contained allopeptide reactive T cells. The frequency of acute rejection episodes, occurring one month following the LDA test, was significantly higher when allopeptide reactive T cells were present in the circulation, than when they were absent (p,0.001) (FIG.,7). I1-2 expanded T cells from 20 healthy controls showed no proliferation to mixtures of synthetic allopeptides.

Fourteen recipients had concomitant PBMC and biopsy specimens. Tests of linear trend revealed a significant correlation between allopeptide reactivity in the periphery (p,0.001) and graft (p,0.002), and progression of the rejection process (Table 3).

TABLE 3
Relationship Between T Cell Reactivity to Donor
Allopeptide and Clinical Status

| | | Time of LDA Testing | | | | |
|---|---|---|---|---|---|---|
| | | | | | Quiescence | |
| Spec- | LDA | Before | Concom- | Resolv- | No | |
| imen | Results | Onset | mitant | ing | rejection | Total |
| PBMC | Positive | 18 | 22 | 6 | 3 | 49 |
| | Negative | 10 | 3 | 11 | 47 | 71 |
| | Total | 28 | 25 | 17 | 50 | 120 |
| | | | p. < 0.001 | | | |
| Graft | Positive | 2 | 6 | 1 | 1 | 10 |
| | Negative | 1 | 1 | 0 | 11 | 13 |
| | Total | 3 | 7 | 1 | 12 | 23 |

p < 0.002

Table 3. IL-2 expanded T cells from the peripheral blood and graft biopsy tissue obtained at various times following transplantation were tested in LDA for reactivity to donor allopeptides. The number of specimens showing allopeptide-reactive T cells is indicated. The relationship between T cell reactivity to donor allopeptides and clinical status was calculated using the test of linear trend (Dixon et al., 1990).

The presence of allopeptide reactive cells in the circulation was predictive of rejection in 18 of 28 cases (64%). Although at the time of LDA testing, these patients showed no histopathologic evidence of rejection, the subsequent biopsy (performed within 1-4 weeks) showed rejection grade 1B, 2 or 3. The early appearance of allopeptide-reactive Th cells in the circulation suggests that these cells play a role in initiating rejection by producing the lymphokines required for the generation of CTL, DTH and inflammatory responses. LDAS performed at the time of rejection revealed the presence of T cells activated by the indirect pathway in most PBMC (88%) specimens tested. During resolving rejection, which is characterized histologically by a lesser degree of graft damage than seen before rejection treatment (Billingham, 1995), only 35% of PBMC samples studied showed allopeptide reactive T cells. Ninety-four percent of LADS were negative in patients with no histologic evidence of rejection for a period of one month, indicating that quiescence was related to the absence of allopeptide reactive T cells (Table 3). The decreased incidence of positive LDA after treatment of acute rejection suggests that the indirect recognition pathway is susceptible, at least in part, to inhibition by conventional immunosuppressive drugs (methylprednisolone and cyclosporine), consistent with other studies (Sawyer et al., 1993; and Gallon et al., 1995).

Frequency and HLA-DR Specificity of Allopeptide Reactive T Cells

The frequency of allopeptide-reactive T cells was 10-50 fold higher within the population of T cells infiltrating the graft than within the subset of peripheral blood T cells selectively expanded in medium with rIL-2 (Table 4).

TABLE 4
Frequency of Donor-Specific, Self-MHC Restricted T Cells in the Graft and in the Periphery

| | | | Time of | | Frequency of Allopeptide Specific T Cells | | | |
|---|---|---|---|---|---|---|---|---|
| | | | Study (Wks. | | Graft | | Periphery | |
| | HLA-DR | | Post Trans- | Histologic | DR1 | DR3 | Dr1 | Dr3 |
| Patient | Recipient | Donor | plantation | Grade | Peptide | Peptide | Peptide | Peptide |
| W.O. | 0701/0803 | 0101/0301 | 6 | 1A | 1.1 × 10⁻⁴ | 0 | 5.7 × 10⁻⁶ | 0 |
| | | | 8 | 3A | 3.7 × 10⁻⁴ | 0 | 3.6 × 10⁻⁶ | 0 |
| | | | 12 | 1A | 0 | 0 | 0 | 0 |
| | | | | | DR1 | DR14 | DR1 | DR14 |
| | | | | | Peptide | Peptide | Peptide | Peptide |
| I.P. | 0404/1104 | 0101/1401 | 6 | 2 | ND | ND | 7.4 × 10⁻⁶ | 0 |
| | | | 8 | 3A | 4.3 × 10⁻⁴ | 0 | 8.0 × 10⁻⁶ | 0 |
| | | | 12 | 1B | 3.3 × 10⁻⁴ | 0 | 6.5 × 10⁻⁶ | 0 |
| | | | | | DR7 | DR3 | DR7 | DR3 |
| | | | | | Peptide | Peptide | Peptide | Peptide |
| D.R. | 1101/1101 | 0301/0701 | 2 | 1A | 0 | 0 | 0 | 0 |
| | | | 13 | 1B | 2.6 × 10⁻⁴ | 0 | 4 × 10⁻⁶ | 0 |
| | | | 14 | 1B | ND | ND | 3.1 × 10⁻⁶ | 0 |
| | | | 16 | 2 | 2.7 × 10⁻⁴ | 0 | 6.0 × 10⁻⁶ | 0 |
| | | | 17 | 0 | 0 | 0 | 0 | 0 |
| | | | | | DR8 | DR10 | DR8 | Dr10 |
| | | | | | Peptide | Peptide | Peptide | Peptide |
| B.M. | 0301/0701 | 08/10 | 2 | 3A | 0.9 × 10⁻⁴ | 0 | 1.8 × 10⁻⁶ | 0 |
| | | | 8 | 0 | 0 | 0 | 0 | 0 |

This suggests that T cells activated via the indirect allorecognition pathway participate actively in acute allograft rejection.

Of particular interest was the finding that although 19 of the heart recipients included in these studies were mismatched from the donor for two HLA-DR antigens, self-MHC restricted Th reactivity was directed against only one of the donor's HLA-DR antigens in 15 of these patients.

LDA studies of allopeptide reactivity showed that this biased T cell recognition of a single donor DR antigen occurred both in the periphery and in the graft (Table 4). Because the frequency of allopeptide reactive Th cells infiltrating the graft during rejection was 10-50 fold higher, compared to the periphery, it is unlikely that the failure of recipient T cells to recognize the second mismatched HLA-DR antigen was due to the low frequency of the corresponding TCRs and/or to the insensitivity of the assay system. It is more plausible that there was differential processing and competition between allopeptide for occupancy of MHC-class II antigens on host APCs and that a single dominant allopeptide reached the stimulatory threshold (Benichou and Fedoseyeva, 1996; Barbey et al., 1995; Fling et al., 1994; and Morris et al., 1994).

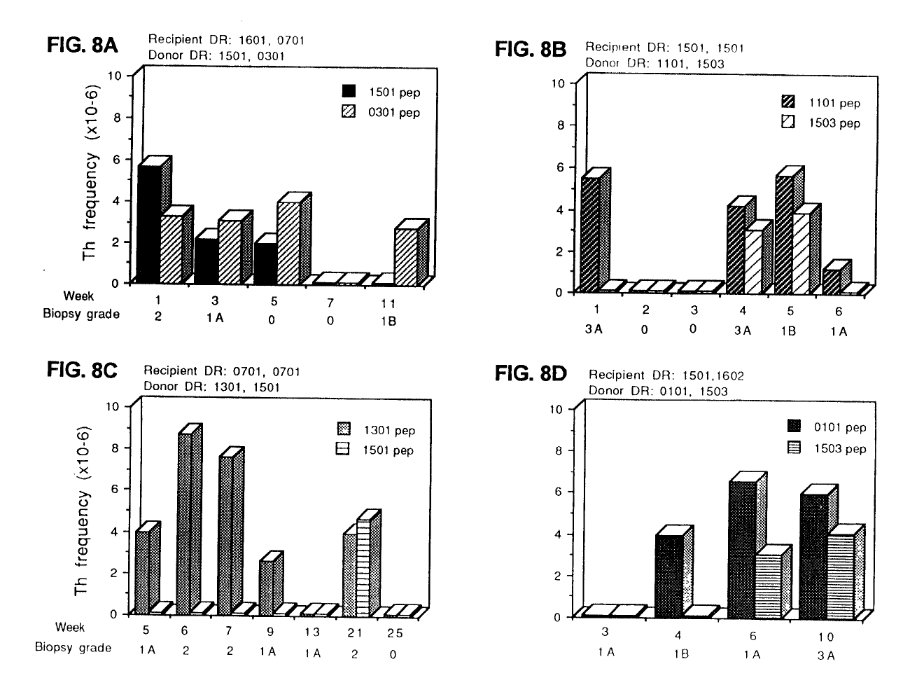

However, the T cell response to donor antigens during allograft rejection appears to be a dynamic process. Thus, in four patients who displayed multiple rejection episodes, indirect allorecognition of both donor DR antigens occurred at various times following transplantation (FIG. 8). In only one of these patients (FIG. 8A), who has mismatched from the donor by HLA-DRβ*1501 and 0301, LDA study of the peripheral blood showed Th reactivity against both DR15 and DR3 peptides throughout the first rejection episode. A second episode, occurring 11 weeks following transplantation was accompanied by reactivity against DR3 but not against DR15 peptides. In the remaining 3 patients alloreactivity was directed against a single donor DR antigen during the first episode, yet the response spread to include the other DR antigen during subsequent episodes of acute rejection (FIGS. 8B, 8C, 8D).

To identify the determinant(s) which T cells recognize during rejection, allopeptide-reactive T cells from LDA studies were expanded in medium containing rIL-2, autologous APCs and the mixture of stimulatory peptides corresponding to the sensitizing HLA-DR antigen. The resulting T cell lines were tested for reactivity to each component of the mixture in the presence of hemi-allogeneic APCs sharing with the recipient one HLA-DR antigen. These experiments showed that although six of the eight T cell lines studied were obtained from HLA-DR heterozygous recipients, allopeptide reactivity was restricted by a single HLA-DR element of the host (Table 5). As indicated above, the response was directed against the dominant determinant of a single HLA-DR antigen, even when the donor was mismatched from the recipient by two HLA-DR alleles (Table 5, patients B. C., I. P., B. R., X. J., F. J., H. M.).

TABLE 5
Determination of Host Restriction Element and Dominant Allopeptide

| | | | | DR Peptide | ³H-TdR Incorporation (cpm) | | |
|---|---|---|---|---|---|---|---|
| Patient | Recipient | HLA-DR Donor | APC | Specificity | Peptide 1-19 | Peptide 21-39 | Peptide 62-80 |
| B.C. | 0701/1101 | 0101/0401 | 0701/0701 | 0101 | 1,764 | 1,660 | 1,416 |
| | | | 1101/1201 | | 1,649 | 35,027 | 1,770 |
| I.P. | 0404/1104 | 0101/1401 | 0404/1501 | 0101 | 560 | 700 | 471 |
| | | | 1101/1502 | | 137 | 18,360 | 860 |
| K.R. | 0101/1501 | 0301/0301 | 0101/0401 | 0301 | 5,075 | 112 | 293 |
| | | | 1501/0401 | | 108 | 117 | 192 |
| K.T. | 0404/0701 | 0401/0404 | 0404/1501 | 0401 | 1,917 | 1,926 | 1,403 |
| | | | 0701/0701 | | 1,942 | 1,741 | 22,953 |
| B.R. | 0701/0701 | 1301/1501 | 0701/0701 | 1301 | 43,867 | 2,538 | 1,804 |
| X.J. | 0101/0401 | 1101/1501 | 0101/1201 | 1501 | 2,348 | 2,203 | 2,009 |
| | | | 0401/1401 | | 49,368 | 2,102 | 1,728 |
| F.J. | 1104/1401 | 0901/1501 | 1104/1502 | 1501 | 5,461 | 137 | 103 |
| | | | 1401/0401 | | 115 | 131 | 110 |
| H.M. | 1501/1501 | 1101/1503 | 1501/1501 | 1503 | 245 | 13,286 | 259 |

Table 5

T cells, reacting in LDA to donor allopeptides, were expanded in medium containing the stimulatory peptide mixture (1 μM of each peptide), rlL-2 (20 μ/ml) and autologous APCs (1×10⁶). The resulting T cell lines were tested for reactivity to each component of the mixture in the presence of APCs sharing with the recipient one HLA-DR antigen.[³II] TdR incorporation in 3-day blastogenesis assay was measured by liquid scintillation counting. Mean cpm of triplicate reactions is indicated. The SD to the mean was less than 10%.

Mapping of the dominant determinant of the DRβ*0101 molecules recognized in vivo by DRβ*1101 recipients confirmed previous in vivo studies, identifying peptide 21-39 as the dominant peptide. Seven additional dominant determinants were identified (Table 5). Peptide 21-39 of DRβ*0101 molecule is also the dominant T cell epitope recognized by DRβ*1104 responders. The dominant epitope DRβ*0301 protein presented by DRβ*0101 molecule is the dominant determinant presented in the context of DRβ*0701. Peptide 1-19 of the DRβ*1301 protein is the dominant determinant recognized by T cells in the context of DRβ*0701. Peptide 1-19 of the DRβ*1501 molecule is the dominant determinant which T cells recognize in the context of DRβ*0401 and DRβ*1104. Finally, peptide 21-39 of DRβ*1503 is the dominant determined recognized by DRβ*1501 responders.

Based on previous studies showing that when dominant allopeptides are used at high concentration they inhibit specifically the indirect recognition response it is expected that these newly mapped determinants may also induce high zone tolerance to the corresponding MHC molecule (Liu et al., 1995).

Discussion

It is currently accepted that both the direct and indirect pathway of allorecognition are involved in allograft rejection. The frequency of T cells which directly recognize intact allo-MHC molecules on the surface of target cells is at least 100 fold higher than that of T cells recognizing allopeptides processed and presented by self MHC (Liu et al., 1993). This finding suggests that the direct pathway of allorecognition dominates the rejection response during the early post-transplantation period when MHC-class II antigens expressed on donor dendritic cells vigorously stimulate the T helper cell response of the host.

However, the role of direct allorecognition seems to diminish after passenger leukocytes of donor origin have left the graft (Mason et al., 1981; and Lechler and Batchelor 1982). Thus, experiments in rats have shown that adoptive transfer of alloreactive T cells, primed by the direct pathway, results in rejection of kidney allografts containing donor dendritic cells but has no effect on grafts depleted of passenger leukocytes (Braun et al., 1993).

Furthermore, although lymphokines produced by T helper cells, notably interferon γ, induce MHC class II expression on endothelial and epithelial cells of the graft, such defective non-professional APCs anergize rather than stimulate alloreactive T cells (Benichou and Fedoseyeva, 1996; and Vidard et al., 1992). In contrast to the direct recognition pathway which depends on the presence in the graft of professional APCs of donor origin, the indirect pathway is stimulated by allopeptides presented by professional APCs (dendritic cells, macrophages) of host origin. Because of the continuous supply of host APCs which have processed donor allopeptides, self-MHC restricted Th cells may perpetuate rejection, generating help for activation of CTL and alloantibody production. This may eventually result in chronic rejection, a phenomenon which claims the function of almost 50% of all organ allografts within 5 years following transplantation. The prevention of this irreversible process may require the development of novel strategies for suppression of the indirect allorecognition pathway. The exquisite specificity of self-MHC restricted T cells for the dominant determinant of allogeneic MHC molecules raises the possibility of using high doses of dominant peptide antigens for tolerance induction or of designing non-immunogenic analogs of the wild-type peptide which act as MHC blockers or TCR antagonists.

However, the design of therapeutic protocols bases on the use of peptides awaits a detailed understanding of the relationship between self-MHC restricted T cell alloreactivity and rejection.

The present study describes for the first time the kinetics of indirect allorecognition in patients with organ allografts. These investigations demonstrate that in heart transplantation the development and progression of acute rejection is strongly associated with the appearance both in the graft and in the recipient's circulation of T cells which react to donor HLA-DR peptides. Because the presence of allopeptide reactive T cells in the circulation was predictive of rejection, indirect alloreactivity seems to be the cause rather than the consequence of acute rejection. It is likely that following alloantigen recognition allopeptide primed T cells infiltrate the graft and secrete lymphokines which promote the generation of CTL, DTH and inflammatory responses characteristic of acute rejection. During resolving rejection and quiescence self-MHC restricted alloreactive T cells disappeared from the circulation and graft tissue, in a pattern consistent with the concept that they play an active role in the acute rejection process. Although the frequency of allopeptide reactive T cells was 10-50 fold lower in the periphery than in the graft, the finding that they were detectable in the circulation before rejection could be recognized histologically on graft biopsies opens the way to the development of non-invasive procedures for early prediction of rejection.

Of particular interest was the finding that in recipients transplanted with heart allografts mismatched for two HLA-DR antigens primary episodes of acute rejection appeared to be triggered by only one of the donor's HLA-DR antigens. Study of Th reactivity in the circulation and in the graft showed that the response was specific for a dominant peptide derived from the polymorphic region of one of the donor's HLA-DR antigen. Although most patients were HLA-DR heterozygous Th reactivity to donor allopeptide was restricted in all cases by a single HLA-DR element of the host.

This biased recognition of a sole allodeterminant in HLA-DR heterozygous donor-recipient combinations may reflect differential antigen processing and selection of specific peptides, a phenomenon presumed to be controlled by an allelic polymorphism in linkage disequilibrium with some MHC-class II genes (Benichou and Fedoseyeva, 1996; Barbey et al., 1995; Fling et al., 1994; and Morris et al., 1994). By contributing to the selection of MHC peptides available for presentation on MHC-class II molecules, this allelic polymorphism may play an essential role in T cell alloreactivity.

However, the specificity of T cell responses to donor antigens may change during depression of rejection since in a few patients with multiple acute rejection episodes alloreactivity spread from one donor HLA-DR to the second DR antigen. This finding has important implication for the design of antigen-specific immunotherapies for the treatment of acute and chronic rejection.

It is possible that inflammation within the graft, interferon y production, tissue damage and upregulation of MHC expression may contribute to intermolecular antigen spreading following a rejection episode. The observation that T cell specificities appear to change during the relapsing-remitting course of the alloimmune response is consistent with studies on autoimmune diseases (Benichou and Fedoseyeva, 1996; and McRae et al., 1995) as well as with the finding that the antibody response of transplant patients spreads from one alloantigen to another during rejection (Suciu-Foca et al., 1991).

Mapping of dominant T cell allodeterminants is essential for the design of inhibitory peptide. Further immunogenetics analysis will be required to determine whether peptides which are dominant in the context of one HLA-DR restrictive element are cryptic when presented by the other haplotype. Although during the course of these follow-up studies no intramolecular spreading of epitopes was observed, it remains possible that determinants which are cryptic when presented by one type of APC (dendritic cells for example) become dominant when presented by other APCs (such as B cells) (Benichou and Fedoseyeva, 1996; and Suciu-Foca et al., 1991). It will be important to determine whether alloreactive B cells, which participate in antibody-mediated chronic rejection, activate T cell reactivity to such cryptic allopeptides.

Taken together, the data indicate that following transplantation T cells recognize processed forms of donor HLA proteins associated with a host HLA-DR restriction element. The response of in vivo activated T cells is limited to a single dominant determinant of an allogeneic MHC molecule consistent with previous studies on human T cells allo-activated in vitro with soluble HLA-DR proteins (Liu et al., 1995), and on such T cells from mice undergoing skin allograft rejection (Benichou and Fedoseyeva, 1996).

Because T cell receptor Bβ gene usage in response to the dominant allopeptide is also limited (Liu et al., 1992; and Liu et al., 1993), selective immune intervention such as treatment with anti-TCR antibodies or induction of tolerance to the dominant determinant may represent viable options for specific suppression of indirect recognition. Treatment to block the indirect recognition pathway may prevent the initiation and amplification of transplant rejection, particularly after professional APCs of donor origin have left the graft, rendering it less vulnerable to direct allorecognition.

### REFERENCES

Auchincloss, H. Jr., R. Lee, S. Shea, J. S. Markowitz, M. J. Grusby, and L. H. Glimcher (1993) The role of “indirect” recognition in initiating rejection of skin grafts from major histocompatibility complex class II-deficient mice. Proc. Natl. Acad. Sci. USA 90:3373-3377.

Barbey, C. C. Watts, and G. Corradin (1995) Antigen-processing organelles from DRβ*1101 and DRβ*1104 B cell lines display a differential degradation activity. Eur. J. Immunol. 25:30-36.

Bell, J. I., D. Denney, L. Foster, T. Belt, J. A. Todo, and H. O. McDevitt. 1989 Allelic variation in the DR subregion of the human major histocompatibility complex. Proc. Natl. Acad. Sci. USA 84:6234-6266.

Benichou, G., E. Fedoseyeva, P. V. Lehmann, C. A. Olson, H. M. Geysen, M. McMillan, and E. E. Sercarz. 1994. Limited T cell response to donor MHC peptides during allograft rejection: Implications for selective immune therapy in transplantation. J. Immunol. 153:938.

Benichou, G., P. A. Takizawa, C. A. Olson, M. McMillan, and E. E. Sercarz. 1992. Donor major histocompatibility complex (MHC) peptides are presented by recipient MHC molecules during graft rejection. J. Exp. Med. 175:305-308.

Benichou, G., and E. V. Fedoseyeva (1996) The contribution of peptides to T cell allorecognition and allograft rejection. Intl. Rev. Immunol. 13:231-243.

Billingham, M. D. (1995) Pathology of human cardiac transplantation. In: Cardiovascular pathology. Clinicopathologic correlations and pathogenetic mechanisms. F. S. Schoen and M. G. Gimbrone, Jr., editors, Williams and Wilkins, Baltimore, MD, 108-129.

Bodmer, J. G., S. G. E. Marsh, E. D. Albert, W. F. Bodmer, B. Dupont, H. A. Erlich, B. Mach, W. R. Mayr, P. Parham, T. Sasazuki, G. M. Schreuder, J. L. Strominger, A. Svejgaard, and P. I. Terasaki (1994) Nomenclature for factors of the HLA system. Human Immunol. 41:1-20.

Bradley, J. A. (1996) Indirect T cell recognition in allograft rejection. Intl. Rev. Immunol. 13:245-255.

Braun, Y. M., A. McCormack, G. Webb, and J. R. Batchelor (1993) Mediation of acute but not chronic rejection of MHC-incompatible rat kidney grafts by alloreactive CD4 T cells activated by the direct pathway of sensitization. Transplantation 55:117-182.

Chen, B. P., A. Madrigal, and P. Parham. 1990. Cytotoxic T cell recognition of an endogenous class I HLA peptide presented by a class II HLA molecule. J. Exp. Med. 172:779-788.

Colovai, A. I., E. Renna-Molajoni, R. Cortesini, and N. Suciu-Foca (1996) New approaches to specific immunodulation in transplantation. Intl. Rev. Immunol. 13:161-172.

Critchfield, J. M., M. K. Racke, J. C. Zuniga-Pflucker, B. Cannella, S. S. Raine, J. Goverman, and M. J. Lenardo. 1994. T Cell deletion in high antigen dose therapy of autoimmune encephalomyelitis. Science 263:1139-1143.

Dalchau, R., J. Fangmann, and J. W. Fabre. 1992. Allorecognition of isolated, denatured chains of class I and class II major histocompatibility complex molecules. Evidence for an important role for indirect allorecognition in transplantation. Eur. J. Immunol. 22:669-677.

de Koster, H. S., D. C. Anderson, and A. Termijtelen. 1989. T cells sensitized to synthetic HLA-DR3 peptide give evidence of continuous presentation of denatured HLA-DR3 molecules by HLA-Dp. J. Exp. Med. 169:1191-1196.

Dixon, W. J., M. B. Brown, L. Engelman, R. I. Jennrich (1990) BMDP statistical software manual to accompany the 1990 software release, Berkeley University of California Press.

Fangmann, J., R. Dalchau, and J. W. Fabre. 1992. Rejection of skin allografts by indirect allorecognition of donor class I major histocompatibility complex peptides. J. Exp. Med. 175:1521-1529.

Fisher, P. E., N. Suciu-Foca, E. Ho, R. E. Michler, E. A. Rose, and M. Mancini (1995) Additive value of immunologic monitoring to histologic grading of heart allograft biopsy specimens: implications for therapy. J. Heart & Lung Transplant 14:1156-1161.

Fling, S., B. Arp, and D. Pious (1994) HLA-DMA and -DMB genes are both required for MHC class II peptide complex formation in antigen-presenting cells. Nature 368:554-558.

Gallon, L., B. Watschinger, B. Murphy, E. Akalin, M. H. Sayegh, and C. B. Carpenter. 1995. The indirect pathway of allorecognition. Transplantation 59:612-616.

Gaspari, A. A., M. K. Jenkins, and S. I. Katz (1988) Class II MHC-bearing keratinocytes induce antigen-specific unresponsiveness in hapten-specific THI clones. J. Immunol.

Harris, P. E., Z. Liu, and N. Suciu-Foca. 1992. MHC class II binding of peptides derived from HLA-DR1. J. Immunol. 148:2169-2176.

Howell, M. D., S. T. Winters, T. Olee, H. D. Powell, D. J. Carlo, and S. W. Brosstoff. 1989. Vaccination against experimental allergic encephalomyelitis with T cell receptor peptides. Science 246:668-670.

Krensky, A. M., and C. Clayberger, 1995. HLA-derived peptides as novel immunotherapeutics. Clin. Immunol. Immunopath. 75:112-116.

Lechler, R. I., and J. R. Batchelor. 1982. Restoration of immunogenicity to passenger cell depleted kidney allografts by the addition of donor strain dendritic cells. J. Exp. Med. 155:31-41.

Lechler, R. I., G. Lombardi, J. R. Batchelor, N. Reinsmoen, and F.H. Bach (1990) The molecular basis of alloreactivity. Immunol. Today 11:83-88.

Lechler, R. I., T. Heaton, L. Barber, V. Ball, J. R. Batchelor, and G. Lombardi (1992) Molecular mimicry by major histocompatibility complex molecules and peptides accounts for some alloresponses. Immunol. Lett. 34:63-70.

Lee, R. S., M. J. Grusby, L. H. Glimcher, H. J. Winn, and H. Auchincloss, Jr. 1994. Indirect recognition by helper cells can induce donor-specific cytotoxic T lymphocytes in vivo. J. Exp. Med. 197:865-872.

Liu, Z., N. Brausntein, and N. Suciu-Foca. 1992. T cell recognition of allo-peptides in context of syngeneic MHC. J. Immunol. 148:35-40.

Liu, Z., Y. K. Sun, Y. P. Xi, A. Maffei, E. Reed, P. E. Harris, and N. Suciu-Foca. 1993. Contribution of direct and indirect recognition pathway to T cell alloreactivity. J. Exp. Med. 177:1643-1650.

Liu, Z., Y. K. Sun, Y. P. Xi, B. Hong, P. E. Harris, E. Reed, and Suciu-Foca. 1993. Limited usage of TCR Vβ genes by allopeptide specific T cells. J. Immunol. 150:3180-3186.

Liu, Z., P. Harris, A. I. Colovai, E. F. Reed, A. Maffei, and N. Suciu-Foca (1995) Suppression of the indirect pathway of T cell reactivity by high doses of allopeptide. Autoimmunity 21:173-184.

Lo, D., L. C. Burkly, R. A. Flavell, R. D. Palmiter, and Brinster, R. L. (1989) Tolerance in transgenic mice expressing class II major histocompatibility complex on pancreatic acinar cells. J. Exp. Med. 170:87-104.

Mason, D. W., C. W. Pugh, and M. Webb (1981) The rat mixed lymphocyte reaction: roles of a dendritic cell in intestinal lymph and T-cell subsets defined by monoclonal antibodies. Immunol. 44:75-87.

Matzinger, P. and M. J. Bevan (1977) Hypothesis: Why do so many lymphocytes respond to major histocompatibility antigens? Cell Immunol. 29:1-15.

McRae, B. L., C. L. Vanderlugt, M. C. Cal Canto, and S. D. Miller (1995) Functional evidence for epitope spreading in the relapsing pathology of experimental autoimmune encephalomyelitis. J. Exp. Med. 182:75-85.

Morris, P., J. Shaman, M. Attaya, S. Goodman, C. Bergman, J. Monaco, and E. Mellins (1994) An essential role for HLA-DM in antigen presentation by class II major histocompatibility molecules. Nature 368:551-553.

Parker, K. E., R. Dalchau, V. J. Fowler, C. A. Priestly, C. A. Carter, and J. W. Fabre. 1992. Stimulation of CD4⁺ T lymphocytes by allogeneic MHC peptides presented on autologous antigen-presenting cells. Evidence of the indirect pathway of allorecognition in some strain combinations. Transplantation 53:918-924.

Parker, D. C. (1993) T cell dependent B cell activation. Ann. Rev. Immunol. 11:331-360.

Sawyer, G. J., R. Dalchau, and J. W. Fabre (1993) Indirect T cell allorecognition: a cyclosporin A resistant pathway for T cell help for antibody production to donor MHC antigens. Transplant. Immunol. 1:77-81.

Sayegh, M. H. and C. B. Carpenter (1996) Role of indirect allorecognition in allograft rejection. Intl. Rev. Immunol. 13:221-229.

Sayegh, M. H., S. J. Khoury, W. W. Hancock, H. L. Weiner, and Carpenter, C. B. 1992. Induction of immunity and oral tolerance with polymorphic class II major histocompatibility complex allopeptides in the rat. Proc. Natl. Acad. Sci. USA 89:7762-7766.

Sercarz, E. E., P. V. Lehmann, A. Ametani, G. Benichou, A. Miller, and K. Moudgil. 1993. Dominance and crypticity of T cell antigenic determinants. Annu. Rev. Immunol. 11:729-766.

Sheirle, A., B. Takacs, L. Kremer, F. Marin, And F. Sinigaglia. 1992. Peptide binding to soluble HLA-DR4 molecules produced by insect cells. J. Immunol. 149:1994-1999.

Sherman, L. A. and S. Chattopadhyan. 1993. The molecular basis of allorecognition. Annu. Rev. Immunol. 11:385-402.

Shoskes, D. A., and K. J. Wood. 1994. Indirect presentation of MHC antigens in transplantation. Inmunology Today 15:32-38.

Steinman, R. M., and M. D. Witmer (1978) Lymphoid dendritic cells are potent stimulators of the primary mixed leukocyte reaction in mice. Immunol. 75:5132-5136.

Suciu-Foca, N., E. Reed, V. D. D'Agati, E. Ho., D. J. Cohen, A. I. Benvenisty, R. McCabe, J. M. Brensilver, D. W. King, and M. A., Hardy. 1991. Soluble HLA antigens, anti-HLA antibodies and anti-idiotypic antibodies in the circulation of renal transplant recipients. Transplantation 51:593-601.

Suciu-Foca, N., E. Reed, C. Marboe, Y. P. Xi, Y. K. Sun, E. Ho., E. A. Rose, K. Reemtsma, and D. W. King. 1991. Role of anti-HLA antibodies in heart transplantation. Transplantation 51:716-724.

Suzuki, G., Y.Kawase, S. Koyasu, I. Yahara, Y. Kobayashi, and R. H. Schwartz. 1988. Antigen-induced suppression of the proliferative response of T cell clones. J. Immunol. 140:1359-1365.

Vandenbark, A. A., G. Hashim, and H. Offner. 1989. Immunization with a synthetic T cell receptor V-region peptide protects against experimental autoimmune encephalomyelitis. Nature 341:541-544.

Vidard, L., K. R. Rock, and B. Benacerraf (1992) Heterogeneity in antigen processing of different types of antigen presenting cells. J. Immunol. 149:1905-1911.

Watschinger, B., L. Gallon, C. B. Carpenter, and M. H. Sayegh (1994) Mechanisms of allo-recognition: Recognition by in vivo-primed T cells of specific major histocompatibility complex polymorhisms presented as peptides by responder antigen-presenting cells. Transplantation 57:572-576.

Winters, G. L., 1991. The pathology of heart allograft rejection. Arch. Pathol. Lab. Med. 115:269-273.

Zhang, J., S. Markovic-Leese, B. Lacet, F. Raus, H. L. Weiner, and D. A. Hafler. 1994. Increased frequency of interleukin 2-responsive T cells specific for myelin basic protein and proteolipid protein in peripheral blood and cerebrospinal fluid of patients with multiple sclerosis. J. Exp. Med. 179:973-984.

What is claimed is:

1. A method for monitoring allograft rejection in a transplant recipient which comprises:
(a) obtaining peripheral blood lymphocytes from the recipient;
(b) incubating the lymphocytes with at least one synthetic allopeptide which corresponds to an immunogenic hypervariable epitope of a mismatched donor HLA-DR antigen under suitable conditions;
(c) contacting the incubate with a marker for T-cell activation to determine whether or not the incubate contains activated T-cells, thereby monitoring allograft rejection in the recipient.

2. The method of claim 1, wherein the synthetic allopeptide comprises at least a portion of a peptide chosen from the group: DRβ1*0101, DRβ1*0102, DRβ1*0301, DRβ1*0302, DRβ1*0401, DRβ1*0102, DRβ1*0403, DRβ1*0404, DRβ1*0405, DRβ1*0407, DRβ1*0408, DRβ1*0701, DRβ1*0801, DRβ1*0802, DRβ1*0803, DRβ1*0804, DRβ1*0901, DRβ1*1001, DRβ1*1101, DRβ1*1102, DRβ1*1104, DRβ1*1201, DRβ1*1301, DRβ1*1302, DRβ1*1303, DRβ1*1401, DRβ1*1402, DRβ1*1502, DRβ1*1503, DRβ1*1601, or DRβ1*1602.

3. The method of claim 1, wherein the allograft comprises a heart, a kidney, a liver, skin, bone, bone marrow, an eye, hair, or a lung.

4. The method of claim 1, wherein determining whether or not the incubate contains activated T-cells comprises detecting T-cell blastogenesis, detecting lymphokine production by the T-cell or detecting expression of an activation marker by the T-cell.

5. The method of claim 4, wherein the activation marker is a monoclonal antibody or an antigen capable of measuring specific early T cell activation in response to a donor antigen.

6. The method of claim 5, wherein the monoclonal antibody is CD69, CD64, LDA1, NDA4 or γIF.

7. The method of claim 1, wherein the synthetic allopeptide comprises a portion of a hypervariable region of an HLA-DR molecule.

8. The method of claim 7, wherein the portion of the HLA-DR hypervariable region comprises amino acids 1-19, amino acids 20-40 or amino acids 41-80.

9. The method of claim 1, wherein the synthetic allopeptide comprises:
amino acids 21-39 of a DRβ1*0101 molecule wherein the recipient expresses a DRβ1*1104 haplotype or a DRβ1*1101 haplotype;
amino acids 1-19 of a DRβ1*0301 molecule wherein the recipient expresses a DRβ1*1101 haplotype;
amino acids 62-80 of a DRβ1*0401 molecule wherein the recipient expresses a DRβ1*0701 haplotype;
amino acids 21-42 of DRβ1*1101 molecule wherein the recipient expresses a DR3 haplotype;
amino acids 21-40 of DRβ1*0401 molecule wherein the recipient expresses a Drβ1*0701;
amino acids 1-19 of a DRβ1*1301 molecule wherein the recipient expresses a DRβ1*0701 haplotype;
amino acids 1-19 of a DRβ1*1501 molecule wherein the recipient expresses a DRβ1*0401 haplotype and a DRβ1*1104 haplotype, or
amino acids 21-29 of a DRβ1*1503 molecule wherein the recipient expresses a DRβ1*1501 haplotype.

10. A method for treating allograft rejection in a transplant recipient which comprises administering to the recipient a mixture of soluble MHC derived from the recipient and at least one synthetic allopeptide which corresponds to an immnogenic hypervariable epitope of a mismatch donor HLA-DR antigen thereby treating allograft rejection in the recipient.

11. A method for treating allograft rejection in a recipient which comprises:
(a) determining whether or not allopeptide reactive T-cells are present in the recipient, and if such cells are present;
(b) administering to the recipient a mixture of soluble MHC derived from the recipient and an allopeptide which comprises ERVRLLERCIYNQE so as to treat allograft rejection in the subject.

12. A method for inhibiting chronic allograft rejection in a recipient which comprises administering to the recipient a peptide antigen corresponding to an epitope on a donor allo-MHC molecule so as to induce tolerance in the recipient and thereby inhibit chronic allograft rejection in the recipient.

13. The method of claim 10, wherein the peptide antigen is administered to the recipient at a dose of about 10 micrograms per one million recipient lymphocyte cells.

14. A vaccine against an allopeptide which comprises a synthetic T-cell receptor peptide which is designed to react against a mismatched MHC-DR allele of an organ donor.

15. The vaccine of claim 11, wherein the mismatched MHC-DR allele is:
DRβ1*0101 wherein the recipient expresses a DRβ1*1104 haplotype or a DRβ1*1101 haplotype;
DRβ1*0301 wherein the recipient expresses a DRβ1*1101 haplotype;
DRβ1*0401 wherein the recipient expresses a DRβ1*0701 haplotype;
DRβ1*1301 wherein the recipient expresses a DRβ1*0701 haplotype;
DRβ1*1501 wherein the recipient expresses a DRβ1*0401 haplotype and a DRβ1*1104 haplotype, or
DRβ1*1503 wherein the recipient expresses a DRβ1*1501 haplotype.
